(19) United States
(12) Patent Application Publication
(10) Pub. No.: US 2025/0043219 A1
(43) Pub. Date: Feb. 6, 2025

(54) GRAPE SKIN COMPOSITIONS AND COMPOUNDS, AND METHODS OF PREPARATION AND USE THEREFOR

(71) Applicant: PHANTASM LIMITED, Hataitai, Wellington (NZ)

(72) Inventor: Joshua RUFFELL, Hataitai, Wellington (NZ)

(73) Assignee: PHANTASM LIMITED, Hataitai, Wellington (NZ)

(21) Appl. No.: 18/716,835

(22) PCT Filed: Nov. 11, 2022
(86) PCT No.: PCT/NZ2022/050143

§ 371 Date: Jun. 5, 2024

(30) Foreign Application Priority Data
Dec. 9, 2021 (NZ) 783291
Dec. 9, 2021 (NZ) 783293

(51) Int. Cl. C12C 12/00; C12C 11/00; C12G 3/02; C12N 9/42; C12N 9/88

(52) U.S. Cl. CPC C12C 12/008; C12C 11/003; C12G 3/02; C12N 9/2445; C12N 9/88; C12Y 302/01021; C12Y 404/01008

(57) ABSTRACT

The present disclosure encompasses concentrates prepared from grape skins, as well as compositions comprising these grape skin concentrates. Also encompassed are methods of preparing these concentrates and compositions, and methods of using the same. Particularly encompassed are methods of using the grape skin concentrates and compositions for preparing various beverages, including fermented beverages, such as beers.

### RELATED APPLICATIONS

This application claims the benefit of New Zealand patent application 783293, filed on 9 Dec. 2021, and New Zealand patent application 783291, filed on 9 Dec. 2021, both of which are incorporated by reference herein in their entirety.

### FIELD

The present disclosure relates to concentrates prepared from grape skins, and compositions comprising these. The present disclosure relates also to methods of preparation and use of such concentrates and compositions, including methods of preparing beverages, including beers and other fermented drinks.

### BACKGROUND

The production of fermented beverages is intrinsically a biotechnological process. The conversion of raw materials into a fermented beverage relies on microbial activity and many different enzymatic reactions.

Traditionally, beer is made from four key ingredients: malted cereals (barley or other), water, hops, and yeast. Cider is traditionally made from fruit juice and yeast, while kombucha is traditionally produced from fruit juice combined yeast and bacterial cultures. Yet, there is also a movement away from traditional fermentation ingredients and methods, and a trend towards more modem techniques.

The worldwide beer, cider, and kombucha industries have grown at an astonishing rate over the past decade, with the number of new manufacturers rapidly increasing. Microbreweries and craft breweries, in particular, have become increasingly numerous.

However, the widespread availability of craft products also means substantial competition between the various producers, and there is a considerable demand for new and improved consumables. Given this, there is a pressing need for new compositions and new methods for preparing fermented beverages.

### SUMMARY

In one aspect, the present disclosure encompasses a method for preparing a composition comprising a grape skin concentrate, the method comprising:
(i) obtaining skin of white wine grapes, such skin having flesh and juice components substantially removed,
(ii) freezing the skin of the white wine grapes,
(iii) thawing the skin of the white wine grapes, and
(iv) drying the skin of the white wine grapes to produce a substantially dried product,
thereby obtaining a composition comprising a grape skin concentrate, and
combining the grape skin concentrate with one or more hops, one or more hops components, one or more yeast, one or more bacteria, and/or one or more enzymes.

In various aspects:

The white wine grapes are aromatic white wine grapes.

The white wine grapes are selected from the group consisting of: white wine grapes from New Zealand, Australia, South Africa, South America, and any combination thereof.

The white wine grapes are selected from the group consisting of: Sauvignon Blanc, Albarino, Chenin Blanc, Colombard, Friulano, Tocai, Gruner Veltliner, Traminer, Verdicchio, Verdejo, Vermentino, Scheurebe, Maccabeo, Gewurztraminer, Riesling, Muscat, Petit Manseng, Pinot Gris, and Tokay grapes, and any combination thereof.

The white wine grapes comprise Sauvignon Blanc grapes.

The white wine grapes consist essentially of Sauvignon Blanc grapes.

The white wine grapes are Sauvignon Blanc grapes.

The substantially removing of (i) comprises crushing or pressing.

The pressing is performed by a pneumatic press or a basket press.

The pressing is performed with a press setting ranging from about 600 to about 800 litres per tonne.

The pressing is performed with a press setting ranging from about 650 to about 720 litres per tonne.

The method includes addition of at least one antioxidant or preservative before or after (i).

The at least one antioxidant is a sulphite.

The at least one antioxidant is ascorbic acid.

The freezing of (ii) comprises blast freezing.

The freezing of (ii) comprises cryomaceration.

The drying of (iv) comprises freeze drying.

The composition comprises a milled form.

The composition comprises a pelleted form.

The composition is mixed with one or more liquids to produce a liquid or semi-liquid form.

The composition comprises a flake or powder.

The powder is prepared with a sieve size of about 4 mm to about 2 mm.

The one or more yeast are selected from the group consisting of: Saccharomyces, Brettanomyces, Kloeckera, Candida, Hanseniaspora, and Pichia yeast.

The one or more yeast include a Pichia kluyveri yeast.

The one or more yeast include a Saccharomyces cerevisiae yeast.

The Saccharomyces cerevisiae yeast is a brewer's yeast.

The one or more yeast include yeast used for conversion of thiol precursors into free thiols.

The one or more bacteria are selected from the group consisting of: Lactobacillus bacteria, Pediococcus bacteria, and any combination thereof.

The one or more bacteria are selected from the group consisting of: Lactobacillus delbrueckii, Lactobacillus plantarum, Pediococcus damnosus, and any combination thereof.

The one or more bacteria include a bacteria used for lactic acid production.

The one or more enzymes include β-lyase.

The one or more enzymes include β-glucosidase.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

The one or more hops include cryogenically prepared hops.

The one or more hops components include lupulin.

In one other aspect, the present disclosure encompasses a composition comprising a grape skin concentrate, the grape skin concentrate being prepared according to a preceding aspect.

In various aspects:

The composition comprises a milled form.

The composition comprises a flake or a powder.

The composition comprises a pelleted form.

The composition comprises a liquid or semi-liquid form.

The composition includes one or more yeast.

The composition includes one or more bacteria.

The composition includes one or more enzymes.

The one or more yeast are selected from the group consisting of: Saccharomyces, Brettanomyces, Kloeckera, Candida, Hanseniaspora, and Pichia yeast.

The one or more yeast include a Pichia kluyveri yeast.

The one or more yeast include a Saccharomyces cerevisiae yeast.

The Saccharomyces cerevisiae yeast is a brewer's yeast.

The one or more yeast include a yeast used for biotransformation of thiol precursors into free thiols.

The one or more bacteria are selected from the group consisting of: Lactobacillus bacteria, Pediococcus bacteria, and any combination thereof.

The one or more bacteria are selected from the group consisting of: Lactobacillus delbrueckii, Lactobacillus plantarum, Pediococcus damnosus, and any combination thereof.

The one or more bacteria include bacteria used for lactic acid production.

The one or more enzymes include β-lyase.

The one or more enzymes include β-glucosidase.

The composition includes one or more hops or hops components.

The powder form includes one or more hops or hops components.

The pelleted form includes one or more hops or hops components.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

The one or more hops include cryogenically prepared hops.

The one or more hops components include lupulin.

In yet one other aspect, the present disclosure encompasses a composition comprising a grape skin concentrate and at least one additional ingredient, the grape skin concentrate being prepared according to a preceding aspect.

In various aspects:

The composition comprises a milled form.

The composition comprises a flake or a powder.

The composition comprises a pelleted form.

The composition comprises a liquid or semi-liquid form.

The at least one additional ingredient is one or more yeast.

The at least one additional ingredient is one or more bacteria.

The at least one additional ingredient is an enzyme.

The at least one additional ingredient is one or more hops or hops component(s).

The at least one additional ingredient is lupulin.

The one or more yeast are selected from the group consisting of: Saccharomyces, Brettanomyces, Kloeckera, Candida, Hanseniaspora, and Pichia yeast.

The one or more yeast include Pichia kluyveri yeast or a Saccharomyces cerevisiae yeast.

The Saccharomyces cerevisiae yeast is a brewer's yeast.

The one or more yeast include a yeast used for biotransformation of thiol precursors into free thiols.

The one or more bacteria are selected from the group consisting of: Lactobacillus bacteria, Pedi coccus bacteria, and any combination thereof.

The one or more bacteria are selected from the group consisting of: Lactobacillus delbrueckii, Lactobacillus plantarum, Pediococcus damnosus, and any combination thereof.

The one or more bacteria include bacteria used for lactic acid production.

The one or more enzymes include β-lyase.

The one or more enzymes include β-glucosidase.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

The one or more hops include cryogenically prepared hops.

The one or more hops components include lupulin.

In one further aspect, the present disclosure encompasses a method of making a fermented beverage, comprising:
introducing a composition comprising a grape skin concentrate during a fermentation and/or prior to initiation of a fermentation, and
carrying out the fermentation, thereby obtaining the fermented beverage,
wherein the composition comprising the grape skin concentrate is prepared according to a preceding aspect, and
the composition includes one or more hops, one or more hops components, one or more yeast, one or more bacteria, and/or one or more enzymes.

In various aspects:

The composition is further introduced post-fermentation.

The composition comprises a milled form.

The composition comprises a flake or powder.

The composition comprises a pelleted form.

The composition comprises a liquid or semi-liquid form.

The method includes introducing one or more yeast during the fermentation and/or prior to initiation of the fermentation.

The method includes introducing one or more bacteria during the fermentation and/or prior to initiation of the fermentation.

The method includes introducing one or more enzymes during the fermentation and/or prior to initiation of the fermentation.

The method includes introducing one or more hops or hops components during the fermentation, prior to initiation of the fermentation, and/or after the fermentation.

The composition includes one or more yeast.

The composition includes one or more bacteria.

The composition includes one or more enzymes.

The composition includes one or more hops or hops components.

The one or more yeast are selected from the group consisting of: Saccharomyces, Brettanomyces, Kloeckera, Candida, Hanseniaspora, Pichia yeast, and any combination thereof.

The one or more yeast include a Pichia kluyveri yeast.

The one or more yeast include a Saccharomyces cerevisiae yeast.

The one or more yeast include a brewer's yeast.

The one or more yeast include yeast used for biotransformation of thiol precursors into free thiols.

The one or more bacteria are selected from the group consisting of: Lactobacillus bacteria, Pediococcus bacteria, and any combination thereof.

The one or more bacteria are selected from the group consisting of: Lactobacillus delbrueckii, Lactobacillus plantarum, Pediococcus damnosus, and any combination thereof.

The one or more bacteria include a bacteria used for lactic acid production.

The one or more hops components include lupulin.

The one or more hops components include hops terpenes.

The one or more hops components include hops esters.

The one or more enzymes include β-lyase.

The one or more enzymes include β-glucosidase.

The one or more hops are selected from the group consisting of: American, Australian, English, German, New Zealand, and South African hops, and any combination thereof.

The one or more hops are selected from the group consisting of: Nelson Sauvin hops (hops identification code 85-03-06), Hallertau Blanc hops (hops identification code 2007/019/008), Tomahawk hops (hops identification code F10), Simcoe hops (hops identification code YCR 14), Summit hops (hops identification code AD24-002), Cascade hops (hops identification code 55187), and any combination thereof.

The one or more hops are selected from the group consisting of: Citra (hops identification code HBC 394), Chinook (hops identification code W-421-38), Loral (hops identification code HBC 291), Mosaic (hops identification code HBC 369), Amarillo (hops identification code VGXP01), Centennial (hops identification code W415-90), Ekuanot (hops identification code HBC 366), Sabro (hops identification code HBC 438), Southern Cross (hops identification code 77-60), Pacific Gem, Pacific Jade (hops identification code Hort 1524), Riwaka (hops identification code 85.6-23), Dr Rudi, Wakatu (hops identification code 77-05), Wai iti (hops identification code Hort 7709), Waimea (hops identification code Hort 3953), Kohatu (hops identification code Hort 3829), Motueka (hops identification code 87.14-20), Green Bullet, Nectaron (hops identification code Hort 4337), Hort 9909, Orbit, Rakau (hops identification code 70-4-9), Pacifica (hops identification code 77-01), Styrian Golding, Strata (hops identification code X331), Galaxy (hops identification code 94-203-008), Calypso (hops identification code 03129), El Dorado, Hallertauer Mittelfrüher, Idaho 7 (hops identification code J-007), Saaz-CZ, Talus (hops identification code HBC 692), Vista (hops identification code 2006009-074), Ella (hops identification code 01-220-06), Enigma, and Vic Secret (hops identification code 00-207-013) hops, and any combination thereof.

The one or more hops include cryogenically prepared hops.

The fermented beverage is an alcoholic, non-alcoholic, or reduced-alcohol beverage.

The fermented beverage is a beer.

The fermented beverage is selected from the group consisting of: barley or other cereal beers, maize beers, millet beers, oat beers, rice beers, rye beers, sorghum beers, and wheat beers, and/or

The fermented beverage is selected from the group consisting of: ales, lagers, pilsners, porters, saisons, and stouts.

The beer is an India Pale Ale beer.

The beer is a New England IPA.

The beer is a hazy IPA.

The fermented beverage is a beer-wine hybrid.

The fermented beverage is a fruit beer.

The fermented beverage is a cider.

The fermented beverage is a kombucha.

In yet a further aspect, the disclosure encompasses a fermented beverage prepared using the method according to a preceding aspect.

In various aspects:

The fermented beverage is an alcoholic, non-alcoholic, or reduced-alcohol beverage.

The fermented beverage is a beer.

The fermented beverage is selected from the group consisting of: barley or other cereal beers, maize beers, millet beers, oat beers, rice beers, rye beers, sorghum beers, and wheat beers.

The beer is selected from the group consisting of: ales, lagers, pilsners, porters, saisons, and stouts.

The beer is an India Pale Ale beer.

The beer is a New England IPA.

The beer is a hazy IPA.

The fermented beverage is a beer-wine hybrid or a fruit beer.

The fermented beverage is a cider.

The fermented beverage is a kombucha.

The foregoing brief summary broadly describes the features and technical advantages of certain embodiments of this disclosure. Further technical advantages will be described in the detailed description and examples that follow. Novel features that are believed to be characteristic of the disclosed subject matter will be better understood from the detailed description when considered in connection with any accompanying figures and examples. However, the figures and examples provided herein are intended to help illustrate this subject matter or assist with developing an understanding of this subject matter, and are not intended to limit its scope.

This disclosure refers specifically to New Zealand provisional application 765291, and International patent application PCT/NZ2021/050090, which are incorporated by reference herein in their entirety.

### BRIEF DESCRIPTION OF THE DRAWINGS

FIG. 1: Thiol precursor levels (C3MH, CG3MH, GC3MH, G3MH) substantially increase in wort samples containing grape skin compositions. Control wort sample omitted the grape skin composition. “750” indicates grape pressing performed at 750 litres per tonne. “720” indicates grape pressing performed at 720 litres per tonne.

FIG. 2: Volatile thiols levels (3MH) substantially increase in beer samples prepared with grape skin compositions. Control wort sample omitted the grape skin composition. “750” indicates grape pressing performed at 750 litres per tonne. “720” indicates grape pressing performed at 720 litres per tonne.

FIG. 3: Volatile thiol levels (3MH) substantially increase in beer samples prepared with grape skin composition (powder) and thiol boosting yeast (Omega Yeast; OYL-402; Cosmic Punch™). Controls included: samples without the grape skin composition and samples that contained a control yeast (Omega Yeast; OYL-011; British Ale V).

FIG. 4: 3MH levels substantially increase in beer samples prepared with grape skin composition (powder), whereas 3M4MP and 4MMP are substantially reduced. Conditions tested: 0% grape skin composition, 100% hops (w/w); 100% grape skin composition, 0% hops (w/w); 20% grape skin composition, 80% hops (w/w); 40% grape skin composition, 60% hops (w/w).

### DETAILED DESCRIPTION

The following description sets forth numerous exemplary configurations, parameters, and the like. It should be recognised, however, that such description is not intended as a limitation on the scope of what is disclosed, but is instead provided as a description of exemplary embodiments.

All references, including patents and patent applications, cited in this specification are hereby incorporated by reference. No admission is made that any reference constitutes prior art. Nor does discussion of any reference constitute an admission that such reference forms part of the common general knowledge in the art, in New Zealand or in any other country.

### Definitions

In each instance herein, in descriptions, embodiments, and examples of the present disclosure, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “consisting essentially of”, as used herein, may refer to the presence of a component in a composition. For example, a concentrate may be at least 80% by weight of the composition, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by weight of the composition (% w/w). For liquids, a concentrate may be at least 80% by volume of the composition volume, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.8%, or at least 99.9% by volume of the composition volume (% v/v).

In the present description, the articles “a” and “an” are used to refer to one or to more than one (i.e., to at least one) of the grammatical object of the article. By way of example, “an element” can be taken to mean one element or more than one element.

Throughout this description, the term “about” is used to indicate that a value includes the standard deviation of error for the method being employed to determine the value, for example, levels of compounds or concentration levels, as described in detail herein. In particular, the term “about” encompasses a 10% to 15% deviation (positive and negative) in the stated value or range, particularly 10% deviation (positive and negative) in the stated value or range.

As used herein, “beer” refers to a fermented beverage prepared from cereal grains, or roots, or other plant parts. Different types beers may be flavoured with one or more types of hops. Exemplary beers include but are not limited to various cereal beers (e.g., beers prepared from malted barley), maize beers, millet beers, oat beers, rice beers, rye beers, sorghum beers, and wheat beers, and any combination of these, as well as various ales, lagers, pilsners, porters, radlers, saisons, and stouts. Beer-wine hybrids are also included, that is, beers brewed in combination with one or more wine grapes. Further included are fruit beers, for example, beers brewed in combination with one or more fruits. Other beers are described in detail herein.

“Concentrate”, for example, in relation to a grape skin, refers to remaining material where the flesh and juice components of the grape have been partly or substantially removed. In addition to this, seeds and/or stems may be partly or substantially removed. Removal of the flesh and juice components may be by one or more of crushing, pressing, drying, and other means. A concentrate may be prepared as a particular composition, for example, as a paste, flake, cake, tablet, powder, plug, or pellet. Various preparations and methods for obtaining these are set out further below.

Utilised herein “fermented beverage” refers to consumable liquids prepared by yeast fermentation. These include but are not limited to beverages such as beers, beer-wine hybrids (classified as a type of beer), fruit beers, and ciders. Kombuchas are also included. The fermented beverage may be an alcoholic beverage, a non-alcoholic beverage, or a reduced-alcohol beverage. In certain circumstances, wines are specifically excluded as fermented beverages.

As noted herein, “freeze drying” refers to a lyophilisation procedure. It will be understood that the terms “freeze drying” does not exclude the use of higher temperatures (i.e., higher than freezing temperatures). For example, higher than freezing temperatures may be used for removing residual moisture during the secondary drying phase for freeze drying procedures. [00169]“Grape” as used herein encompasses any fruit of the genus Vitus, and any hybrid, cultivar, variety, and genetic derivative thereof. Encompassed, specifically, are wine grapes, including Vitis vinifera, and particularly, white wine grapes, including aromatic white wine grapes, such as the particular varietal grapes of Sauvignon wines, including Sauvignon Blanc wines, as well as the varietal grapes of Albarino, Chenin Blanc, Colombard, Friulano (e.g., Tocai), Gruner Veltliner, Traminer, Verdicchio, Verdejo, and Vermentino wines. Also included are Scheurebe, Maccabeo, Gewurztraminer, Riesling, Muscat, Petit Manseng, Pinot Gris, and Tokay wine grapes. Other grapes are described in detail herein. It will be understood that one or more different grapes (or grapes from different regions) may be combined and used.

“Hops” when referring to a beverage additive refers to seed cones (also called flowers or strobiles) of the genus Humulus, and any hybrid, cultivar, variety, and genetic derivative thereof. Specifically encompassed are hops of Humulus lupulus, and particularly, H. l. var. lupulus, H. l. var. lupuloides (e.g., H. americanus), H. l. var. cordifolius, H. l. var. pubescens, and H. l. var. neomexicanus. Exemplary hops include but are not limited to American hops and New Zealand hops, as well as hops from Australian, English, French, Belgian, German, South African, or other sources. Hops of interest are set out in this specification, including specific cultivars. It will be understood that one or more different hops (or hops from different regions) may be combined and used. In addition, wet or dry hops may be utilised, or combinations thereof. Particularly noted are whole leaf hops and components obtained from hops, for example, one or more hops extracts, hops powders, hops pellets, hops plugs, and the like. Cryogenically prepared hops are also noted, i.e., hops cones that are frozen, for example, by liquid nitrogen, and have the lupulin glands separated from the hops leaf bract. Other exemplifications are provided herein.

A “genetic derivative” of a plant refers to offspring, sports, or other cultivars that are obtained from the parent stock. This includes offspring obtained from a genetic cross with the parent, e.g., F1 progeny or F2 progeny.

“Sauvignon Blanc” grapes refer to the specific white wine grapes of Vitis vinifera originating from Western France in the Loire Valley and Bordeaux wine regions, but also grown in notable regions of South Africa, Chile, New Zealand, California, Iran, and the Ukraine. “Sauvignon Blanc” is alternatively known as Sauvignon Jaune, Fumé Blanc, Blanc Fume, Feigentraube, and Muskat-Silvaner, amongst other names.

“Sulphur compounds” refers to various chemical agents associated with aroma and/or flavour in fermented beverages. These include but are not limited to volatile thiols and their precursors (i.e., thiol precursors), as well as furanthiols, mercaptans, esters, phenols, and terpenoids.

“Thiols” refer to sulphur compounds that are analogs of an alcohol. Particular thiols of interest include volatile thiols associated with grapes and/or hops. These include but are not limited to 3-mercaptohexan-1-ol (3MH), 3-mercaptohexyl acetate (3MHA), 4-methyl-4-mercaptopentan-2-one (4MMP), 4-mercapto-4-methylpentan-2-ol (4MMPOH), 3-sulfanyl-4-methylpentan-1-ol (3S4MP), 3-sulfanyl-4-methylpentyl acetate (3S4MPA), 3-mercapto-3-methylbutan-1-ol (3MMB), 3-mercapto-2-methylpropanol (3MMP). Various precursor compounds for volatile thiols are also included (i.e., thiol precursors), for example, S-3-(hexan-1-ol)-L-cysteine (Cys-3MH/C3MH), S-4-(4-methylpentan-2-one)-L-cysteine (Cys-4MMP/C4MMP), S-3-(hexan-1-ol)-glutathione (Glut-3MH/G3MH) and S-4-(4-methylpentan-2-one)-glutathione (Glut-4MMP/G4MMP) for 3MH and 4MMP. In relation to wine grapes, varietal thiols and non-varietal thiols are specifically noted.

As used herein, the term “yeast” refers to one or more strains used for fermentation. Exemplary yeast strains include but are not limited to Saccharomyces, Pichia, Brettanomyces, Kloeckera, Candida, and Hanseniaspora strains. Specifically noted for fermentation are Pichia kluyveri strains and Saccharomyces cerevisiae strains. Other strains are set out herein.

As used herein, the term “bacteria” refers to one or more strains used for fermentation. Exemplary bacterial strains include but are not limited to those of the order Lactobacillales, including, for example, Lactobacillus, Lactococcus, Leuconostoc, Streptococcus, Pediococcus, Oenococcus, Weissella strains. Specifically noted for fermentation are Lactobacillus delbrueckii, Lactobacillus plantarum, Pediococcus damnosus strains. Other strains are set out herein.

### Compositions Comprising Grape Skin Concentrates and Associated Chemical Compounds

The applicant has found that grape skin concentrates may be prepared and used as beneficial compositions for preparing fermented beverages, such as beers, beer-wine hybrids, fruit beers, and ciders, and also kombuchas. In particular, it has been demonstrated that the skins from white wine grapes, and particularly, Sauvignon Blanc varietal grapes, can be processed to obtain a concentrate useful for enhancing desirable aromas and flavours in fermented beverages.

Grapes, and specifically wine grapes, have been shown to contain sulphur compounds that contribute positively to beverage aromas. Notable amongst these are compounds identified as volatile thiols. See, e.g., Coetzee et al, 2012; Roland et al., 2011; Lund et al, 2009; Ribéreau-Gayon et al, 2006; Tominaga et al, 1998. Volatile thiols are therefore of particular interest for the compositions and methods of the present disclosure. Non-limiting examples of these are set out in Table 1, below.

Typically, grapes have low levels of volatile thiols, and instead contain the corresponding precursor compounds. It is during the fermentation process that yeast convert the various precursor compounds into the volatile thiols (i.e., biotransformation takes place), and thereby impart aromas for the final beverage. Non-limiting examples of precursors are set out in Table 2, below.

The three main volatile thiols associated with tropical nuances in wine are 3MH, 3MHA, and 4MMP. 4MMP precursors are found mainly in juice and flesh of grapes. In contrast, 3MH precursors are retained in the skin, particularly when grapes are processed under cooler temperatures and with gentler pressing. With this in mind, the present application proposes methods of preparing and concentrating grape skins while optimising levels of volatile thiols and precursors in these compositions. These compositions, in turn, are useful for enhancing fermented beverages as described in detail herein.

Notably, beers produced with certain hops are also known to contain volatile thiols. Non-limiting examples of these are set out in Table 3, below. Beer hops are also known to produce terpenoids, particularly sulphur-containing terpenoids, which are useful for imparting aromas and flavours. See, e.g., Holt et al., 2019. Therefore, the present disclosure encompasses compositions comprising the grape skin concentrates, as described herein, which may be combined with various hops compositions. Also encompassed are methods of using the various compositions in fermentation. This is set out in detail further below.

Thus, the present disclosure relates generally to a composition prepared from grape skins. Particularly noted are grape skins from wine grapes. Of specific note are grape skins from white wine grapes. While specific compositions prepared from white wine grapes are described herein, that is, Sauvignon Blanc grapes, it is understood that the present disclosure is readily adapted to use with different types of grapes. Amongst exemplary grapes are those that exhibit particular biochemical, taste, and/or aroma similarities to Sauvignon Blanc wine grapes. These include but are not limited to Albarino, Chenin Blanc, Colombard, Friulano (e.g., Tocai), Gruner Veltliner, Traminer, Verdicchio, Verdejo, and Vermentino wine grapes. Other white wine grapes are encompassed, such as those with noted volatile thiol components. These include but are not limited to Chardonnay, Gewurztraminer, Gros Manseng, Koshu, Maccabeo, Muscat, Muscadet, Petit Arvine, Petit Manseng, Pinot Blanc, Pinot Gris, Riesling, Scheurebe, Semilion, Sylvaner, and Tokay wine grapes. Red wine grapes with noted volatile thiol components are also encompassed. Such include but are not limited to Cabernet wine grapes, such as Cabernet Franc and Cabernet Sauvignon, and Carignan, Grenache, Malbec, Merlot, Pinot Noir, and Syrah wine grapes. Further noted are: Airdn, Carmenere, Gamay, Petite Syrah, Petit Verdot, Sangiovese, Tempranillo Ugni Blanc, Viognier, and Zinfandel wine grapes. Any combination of the above may also be used.

In one particular aspect, the composition is prepared from Vitis vinifera Sauvignon wine grapes, for example, Sauvignon Blanc wine grapes, or one or more of the other grapes described herein. In other aspects, one or more genetic derivatives from this plant/these plants may be used. For example, it may be desirable to use F1 or F2 progeny from a genetic cross that includes the parent stock of the plant. Alternatively, any sports or other cultivars obtained from the parent may be used. It may be desirable to source the grapes from Europe, for example, from France or Germany; from America, for example, from California; from Australia; from New Zealand; from South Africa; or from South America, for example, from Chile. A combination of grapes from different sources may also be used.

TABLE 1
Key volatile thiol compounds from wine grapes

| Key compound | Aroma(s) | Structure |
|---|---|---|
| 3-mercaptohexan-1-ol (3MH) (C₆H₁₄OS) also called 3-sulfanylhexan-1-ol (3SH) | passion fruit, grapefruit, gooseberry, guava | 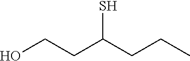 |
| 3-mercaptohexyl acetate (3MHA) (C₈H₁₆O₂S) also called 3-sulfanylhexyl acetate | passion fruit, grapefruit, box tree, gooseberry, guava | 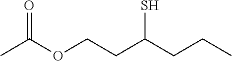 |
| 3-mercapto-3-methylbutan-1-ol (3MMB) also called 3-methyl-3-sulfanylbutan-1-ol | cooked leeks | 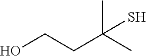 |
| 3-mercapto-2-methylpropanol (3MMP) | broth, sweat | 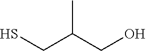 |
| 4-methyl-4-mercaptopentan-2-one (4MMP) (C₆H₁₂OS) also called 4-methyl-4- sulfanylpentan-2-one (4MSP) | box tree, passion fruit, broom, blackcurrant, cat urine | 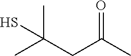 |
| 4-mercapto-4-methylpentan-2-ol (4MMPOH) (C₆H₁₄OS) also called 4-methyl-4-sulfanylpentan-2-ol | citrus zest | 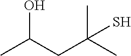 |

TABLE 2
Key volatile thiol precursors

| Precursor | Structure |
|---|---|
| 3MH precursor (E)-hex-2-enal | 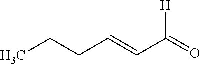 |
| 3MH precursor Glutathionylated precursor (G3MH; also GSH-3SH) | 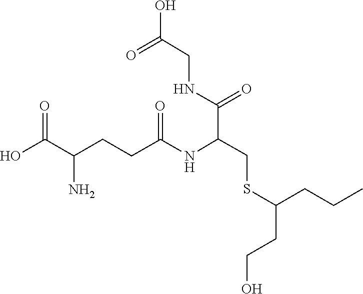 [SMILES: CCCC(CCO)SCC(NC(=O)CCC(N)C(=O)O)C(=O)NCC(=O)O] |
| 3MH precursor Cysteinylated precursor (Cys3MH/C3MH; also Cys-3SH) | 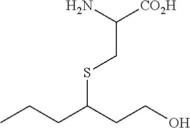 |
| 4MMP precursor Glutathionylated precursor (G4MMP) | 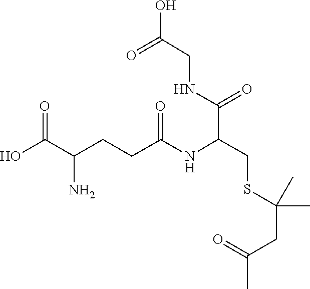 [SMILES: CC(=O)CC(C)(C)SCC(NC(=O)CCC(N)C(=O)O)C(=O)NCC(=O)O] |
| 4 MMP precursor Cysteinylated precursor (Cys4MMP/C4MMP) | 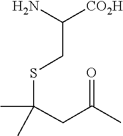 |
| 3MHA precursor 3-mercaptohexan-1-ol (3MH) | 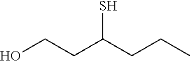 |

TABLE 3
Key volatile thiol compounds from hops

| Key compound | Aroma(s) | Structure |
|---|---|---|
| 3-mercaptohexan-1-ol (3MH) (C₆H₁₄OS) also called 3-sulfanylhexan-1-ol (3SH) | passion fruit, grapefruit, gooseberry, guava | 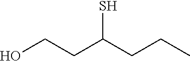 |
| 3-sulfanylpentan-1-ol (3SP) | citrus zest and grapefruit | 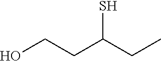 |
| 3-sulfanyl-4-methylpentan-1-ol (3S4MP) | grapefruit and/or rhubarb | 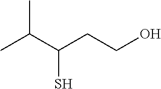 |
| 3-sulfanyl-4-methylpentyl acetate (3S4MPA) | grapefruit, peach, and/or rhubarb | 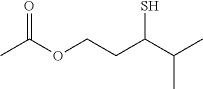 |
| 4-methyl-4-mercaptopentan-2-one (4MMP) (C₆H₁₂OS) also called 4-methyl-4- sulfanylpentan-2-one (4MSP) | box tree, passion fruit, broom, blackcurrant, cat urine | 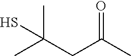 |

The composition is preferably formulated to comprise a grape skin concentrate. It will be understood that the concentrate is prepared from the solid remains of the grapes after crushing or pressing. The concentrate will encompass the skins and, optionally, the seeds and/or stems of the grapes. Accordingly, the composition may be prepared in dried or semi-liquid form. The composition may be formulated, for example, as a flake, powder, pellet, plug, tablet, capsule, syrup, suspension, gel, pomace, puree, paste, or as drops. The composition may be provided in sachet form, for example, a powder or liquid sachet.

In certain aspects, it may be desirable to formulate the grape skin composition into a dried product. Noted formulations include flakes, pellets, plugs, tablets, and capsules. The capsules may include powdered contents. The powder may be provided in free flowing form or as a solid cake. The composition may be provided as a powder for forming a suspension, powder for forming a solution, bulk granules, or bulk powder. In certain circumstances, the dried product may be combined with one or more liquids to re-constitute as a liquid or semi-liquid composition. Other formulations are also possible, as described herein.

As particular examples, the composition of the present disclosure may be combined with one or more hops or one or more ingredients obtained from hops, e.g., one or more hops components. For example, the composition may be formulated as a powder, pellet, or plug, and may include a combination of grape skin concentrate along with hops, hops extract, and/or hops powder. Alternatively, the composition may include a grape skin concentrate combined with whole leaf hops or wet hops. Specifically noted as a hops component is lupulin. As examples, lupulin can be utilised in powder or pellet form, or other dried form. Lupulin enriched hops pellets are specifically noted, e.g., Type 45 (John I Hass). In addition, concentrated lupulin may be provided in the form of cryogenically prepared hops, as described herein. Alternatively, liquid form(s) of lupulin may be utilised, and in the same way liquid form(s) of the grape skin composition may be utilised. Particularly noted are liquid hops products, e.g., Incognito® and Spectrum® (John I Hass), and pourable lupulin fractions. Therefore, the grape skin composition in any form may be combined with any form of lupulin to produce a mixture.

Lupulin can be obtained from one or more hops sources, such as American, Australian, English, German, New Zealand, or South African hops. New Zealand hops, such as Nelson Sauvin, are noted. Particular lupulin compositions include but are not limited to: Citra lupulin powder or pellets (e.g., LupuLN2®), Altus™ lupulin pellets, Amarillo® lupulin pellets, Bravo™ lupulin pellets, Calypso™ lupulin pellets, Cascade lupulin pellets, Centennial lupulin pellets, Comet lupulin pellets, Lemondrop® lupulin pellets, Lotus lupulin pellets, and Sultana™ lupulin pellets. As further exemplifications, C02 extractions and ethanol extractions of hops which produce liquid forms of lupulin may be utilised. Extractions that include hop terpenes (e.g., monoterpenes) are specifically noted. Hops oils are additionally noted. Cryogenically prepared hops (e.g., Cryo Hops® from Yakima Chief Hops), which include a concentrated source of lupulin, are of particular interest. Also of interest are Hopsteiner lupulin pellets and Lupomax® hops pellets from John I Hass.

It will be understood that one type of hops may be used for the disclosed combinations, or different types of hops may be used together. The hops may be obtained from one or more sources, such as American, Australian, English, German, New Zealand, or South African hops. New Zealand hops, such as Nelson Sauvin, are particularly useful. Exemplary hops include but are not limited to: Hallertau Blanc, Tomahawk, Simcoe, Summit, and Cascade hops, as well as Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Saaz-CZ, Talus, Vista, Ella, Enigma, and Vic Secret hops. Various hop blends may be used, for example, Cryo Pop™ blends such as TRI2304CR blended hops (see, e.g., Yakima Chief Hops). The detailed hop variety information will be known and readily identified. See, e.g., International Hop Growers Convention Hop Variety Codes, such as the IHGC hop variety list available from usahops.org; see, also, https://www.yakimachief.com/commercial/hop-varieties.html?product_list_limit=all

The composition may contain one or more additional ingredients, for example, one or more binders, disintegrants, flavours, colours, sweeteners, flow agents, anti-caking agents, sorbents, including one or more antioxidants/preservatives as described herein. Exemplary ingredients include but are not limited to: stearin, magnesium stearate, and stearic acid; saccharides and their derivatives, e.g., disaccharides: sucrose, lactose; polysaccharides and their derivatives, e.g., starches, cellulose or modified cellulose such as microcrystalline cellulose and cellulose ethers such as hydroxypropyl cellulose; sugar alcohols such as isomalt, xylitol, sorbitol and maltitol; proteins such as gelatin; synthetic polymers such as polyvinylpyrrolidone, polyethylene glycol; fatty acids, waxes, shellac, and plant fibres, e.g., corn protein zein; hydroxypropyl methylcellulose; crosslinked polymers, e.g., crosslinked polyvinylpyrrolidone (crospovidone), and crosslinked sodium carboxymethyl cellulose (croscarmellose sodium); sodium starch glycolate; silicon dioxide, fumed silica, talc, and magnesium carbonate.

In certain aspects, the composition of the present disclosure may include a significant level of grape skin concentrate. As exemplifications, the percentages of concentrate in the composition may range from at least 75% to at least 95% grape skin concentrate; or at least 85% to at least 95% grape skin concentrate; or at least 90% to at least 95% grape skin concentrate in the composition (w/w or v/v). The composition may consist essentially of grape skin concentrate as described herein. Where combined with hops (or components obtained from hops, for example, hops powder or hops extract), the percentages of concentrate in the composition may range from at least 15% to at least at least 45% grape skin concentrate; or at least 25% to at least 45% grape skin concentrate; or at least 35% to at least 45% grape skin concentrate in the composition (w/w or v/v). Similarly, the percentages of hops or hops component(s) in the combination may range from at least 15% to at least at least 45% grape skin concentrate; or at least 25% to at least 45% grape skin concentrate; or at least 35% to at least 45% grape skin concentrate in the composition (w/w or v/v).

It is expected that the composition of the present disclosure will include one or more of the beneficial sulphur compounds from wine and/or hops. As noted, volatile thiol compounds are of particular interest for the composition, and specifically, 3MH, 3MHA, and 4MMP, and their precursors. As examples, the composition may include concentrations of one or more volatile thiols (e.g., one or more of 3MH, 3MHA, 4MMP, and/or their precursors) ranging from at least 10 to at least 1000 ng/kg; or at least 40 to at least 800 ng/kg; or at least 100 to at least 700 ng/kg; or at least 500 to at least 600 ng/kg; or concentrations of at least 50 ng/kg, at least 100 ng/kg, at least 500 ng/kg, at least 1000 ng/kg, at least 5000 ng/kg, or at least 10,000 ng/kg. As further examples, concentrations may be ranging from at least 10 to at least 1000 ng/L; or at least 40 to at least 800 ng/L; or at least 100 to at least 700 ng/L; or at least 500 to at least 600 ng/L; or concentrations of at least 50 ng/L, at least 100 ng/L, at least 500 ng/L, at least 1000 ng/L, at least 5000 ng/L, or at least 10,000 ng/L. In certain circumstances, it may be desirable to supplement the sulphur compounds present in the composition. In particular, purified sulphur compounds may be introduced to the composition, including purified volatile thiols such as one or more of 3MH, 3MHA, and 4MMP, and/or their precursors.

In relation to this, it will be understood that other known assays may also be used to analyse the disclosed compositions, and the present disclosure is not limited to one particular assay for bioactive compounds. Components suitable for analysis include but are not limited to sulphur compounds, for example, volatile thiols and their precursors, as well as phenolics, antioxidants, carbohydrates, etc. It will be understood also that the levels of components can be determined for solid forms of the concentrates and also can be readily determined for liquid forms of the concentrates. Exemplary methods include but are not limited to column chromatography, gas chromatography, e.g., gas chromatography-mass spectrometry (GC-MS), liquid chromatography, e.g., high-performance liquid chromatography (HPLC), ultra-performance liquid chromatography tandem mass spectrometry (UPLC-MS/MS), spectrophotometry, e.g., FTIR spectrophotometry, and affinity chromatography.

It has already been noted that the composition of the present disclosure finds particular use for preparing fermented beverages. Methods of preparing and using the disclosed compositions are set out in detail below.

### Methods of Producing Compositions Comprising Grape Skin Concentrates

This disclosure relates generally to a composition prepared from grape skins. Particularly noted are grape skins from wine grapes. Of specific note are compositions prepared using grape skins from white wine grapes, such as Sauvignon Blanc wine grapes. Yet, it will be understood that various types grapes and compositions obtained therefrom are also encompassed by the present disclosure.

As an exemplification of the preparation process, grape skins may be obtained by crushing and/or pressing grapes, and partly or substantially removing the juice and flesh from the grape skins. For example, the practitioner may obtain grape skins (e.g., source or produce), such grape skins having juice and flesh partly or substantially removed. The obtained grape skins can then be frozen or chilled. For example, blast freezing or blast chilling may be used. Individual quick freezing (IQF) may be used. If frozen, the grape skins may be thawed. Following this, the skins are substantially dried to produce a dried product. For example, freeze drying may be used alone or in combination with other drying methods. This dried product, in turn, be further processed to produce a flaked, powdered, or pelletised product. In various aspects, the grape skin concentrate may be prepared to include grape seeds and/or stems, or alternatively, the method can include one or more steps to remove grape seeds and/or stems. Remaining juice after the removal step may be minimised by evaporation, for example, air drying may be used. Remaining juice may also be removed by centrifugation under vacuum. It is preferable to avoid heating during the removal and drying steps.

In initial preparatory stages, the grapes may undergo a pre-treatment process which may include the well known steps of ripening, inspecting, grading, and/or sorting of the grapes. With regard to ripening, it is preferable to use ripe or mature grapes when producing the compositions. Ripeness can be assessed using widely known and used methods in the art. For example, relevant measurements for ripe grapes may range from about 21.7° Bx/12 degree Baumé/93° Oe to about 27.1° Bx/15 degree Baumé/119° Oe. For ripe grapes, particular Brix measurements may be at least about 22° Bx, at least about 23° Bx, at least about 24° Bx, at least about 25° Bx, at least about 26° Bx, or at least about 27° Bx. As exemplifications, grapes may be harvested at a range of about 21 to about 22° Bx. As an alternative, ripeness may correlate to a titratable acidity level between about 0.60-0.99% to about 0.65-0.95%. As a further alternative, ripeness may correlate to a Brix to TA ratio from about 30:1 to about 35:1. Ripeness can be measured prior to picking or processing the grapes. In certain circumstances, overripened grapes may be used.

As part of the processing, the grapes or grape skins may be cleaned and/or sterilised. The grapes may be passed through an assembly having one or more roller brushes for removing dirt or other foreign matter. Conventional washing techniques may be employed. For example, a series of spray nozzles may be used. Wash additives aiding cleansing or reducing the bacteria count may be employed according to local regulations and requirements. As exemplifications, the grapes may be washed by a chlorine wash and/or an ozone impregnated water wash followed by a freshwater rinse.

For processing, the grapes may be conveyed into a hopper. This can be tapered to form a funnel to direct the grapes for pressing. Pressing may be performed with or without a machine. Machine pressing may be accomplished by vertical style pressing, e.g., basket press devices, or horizontal style pressing, e.g., airbag or bladder devices. Other devices include membrane presses, moving head presses, screw presses, impulse presses, and belt presses. Tank presses may also be used. Pressing may be adapted to be performed continuously or in batches. It may be desirable to remove stems prior to pressing by a destemmer/crusher. Alternatively, whole cluster pressing can be used, where the grapes are pressed while still attached to the stems.

The pressing methods may be adapted to apply different levels of force to the grapes as needed. See, e.g., Maggu et al., 2007. As exemplifications, pressures of about 0.4 atm to about 2.4 atm; or about 0.8 atm to about 2 atm; or about 1 atm to about 1.5 atm; or about 0.4 atm, about 0.8 atm, about 1 atm, about 1.2 atm, about 1.4 atm, about 1.8 atm, about 2 atm, about 2.2 atm, or about 2.4 atm may be used for pressing. Pressing may utilise a press setting that ranges from about 500 to about 900 litres per tonne, or about 600 to about 800 litres per tonne, or about 710 to about 760 litres per tonne, or about 740 to about 760 litres per tonne. For example, settings of at least 740, at least 745, at least 750, at least 755, or at least 760 per tonne may be used. Noted settings are at about 720 to about 750 litres per tonne, or about 720, about 730, about 740, about 750, or about 760 litres per tonne.

Pressing may be performed, for example, by a pneumatic press or a basket press. In certain aspects, processing can be relatively gentle (“soft pressing”) compared to conventional pressing techniques. For example, with soft pressing, e.g., pressing set at 650 to 720 litres per tonne or less, there is reduced fragmentation of the skin. In particular, it may be desired that only a minor proportion (generally less than 20%) of skin is fragmented by this process. As an exemplification, a gentle press cycle may be used with an inert gas to avoid oxidisation of the material. Amongst available commercial presses, Bucher Inertys systems may be used, e.g., Bucher XPlus 22 to 80 or Bucher XPert 100 to 450. Computerised systems may be used to control the exact pressure being applied to the grapes and the number of cycles applied. See, e.g., J. Robinson (ed) The Oxford Companion to Wine Third Edition, 2006, pages 285-286, 545-546, 767.

To ensure minimal degradation of ingredients, the preparation process may be performed at a temperature of less than 20° C. In various embodiments, the process is performed at a temperature ranging from −4° C. to 20° C.; or from −1° C. to 15° C.; or from 1° C. to 12° C.; or at about 0° C., about 1° C., about 2° C., about 3° C., about 4° C., about 5° C., or about 6° C. These temperatures may be kept during the preparation process, including the storage of the grapes, prior to being broken open, and during the crushing or pressing process. For example, these temperatures may be kept at least from the point that the grapes have been broken open. Use of such temperatures can reduce oxidation of the grapes and the minimise the use of reducing agents.

Alternatively, or along with these reduced temperature, antioxidants or other preservatives may be added to the grapes or grape skins to retain freshness. As exemplifications, sulphites may be used, e.g., sulphur dioxide. Other examples include but are not limited to sorbic acid, sodium sorbate, potassium sorbate, potassium metabisulphite, citric acid, ascorbic acid, malic acid, tartaric acid, propionic acid, and benzoic acid, for example, in the form of its sodium salt, e.g., sodium benzoate. Any combination of antioxidants/preservatives may be used. Specifically noted are combinations of sulphur dioxide and ascorbic acid. Noted also are combinations of potassium metabisulphite and ascorbic acid. The grape skin concentrate may include, for example, less than 1% of a preservative, e.g., about 0.005% to about 0.5%, or about 0.05% to about 0.15%, or may include about 0.04%, about 0.06%, about 0.08%, about 0.1%, about 0.12%, about 0.14%, about 0.16%, about 0.18%, or about 0.2% of a preservative (w/w).

As specific exemplifications, sulphur dioxide may be added to achieve levels ranging from about 25 to about 55 ppm, or about 30 to about 50 ppm, or about 35 to about 45 ppm in the crushed or pressed grape skins. Preferably, sulphur dioxide levels are less than 300 mg/kg. As other exemplifications, potassium metabisulphite may be added at about 50 to about 70 g per tonne of grapes or pressed grapes, or at about 55 to about 70 g per tonne, or at about 65 to about 68 g per tonne. As further exemplifications, ascorbic acid may be added at about 15 to about 45 parts per tonne of grapes or pressed grapes, or at about 20 to about 35 parts per tonne, or at about 25 to about 35 parts per tonne.

In certain aspects, the antioxidant(s) or other preservative(s) is/are added prior to pressing or crushing, or at least within 10-20 minutes of pressing or crushing. It is also possible to combine antioxidant or preservative agents to increase overall antioxidant activity. To further reduce oxidation, it is possible to use de-oxygenation and inert gas purging. In particular, carbon dioxide and/or nitrogen gas may be introduced during the pressing or crushing process. For example, nitrogen flushing may be used during pressing. It is also possible to add one or more fining agents before, during, or after the pressing or crushing process to protect sulphur compounds. For example, one or more animal or soil components, or one or more synthetic components, may be used as fining agents, including casein, skim milk, egg white (e.g., egg albumin), gelatin, isinglass, bentonite, carbon, and polyvinylpolypyrrolidone (PVPP). In particular aspects, copper may be specifically avoided.

In some circumstances, it may be desirable to adjust the pH of the grape skins or that of the final composition to approximate levels used in fermentation. In particular, it may be useful to obtain a pH range from about 5.0 to about 7.0; or about 5.0 to about 6.5; or about 5.0 to about 6.0; or about 5.0 to about 5.5; or a pH of about 5.1, about 5.2, about 5.3, about 5.4, about 5.5, about 5.6, about 5.7, about 5.8, about 5.9, or about 6.0.

Specific pre-treatments may be utilised. For example, the grape skins may be treated by ultrasonic vibrations and/or pulsed electrical fields. In particular aspects, these treatments may be performed prior to or in lieu of the chilling and/or freezing steps as described herein.

In particular aspects, the grape skins are chilled or frozen directly after removal of the juice and flesh to maintain freshness. Chilling or freezing may also be carried out within 1-2 hours or within 12-24 hours of removal, as needed. Blast chilling and blast freezing are particularly useful methods. As exemplifications, chilling temperatures may range from about 1° C. to about 12° C., about 2° C. to about 10° C., about 4° C. to about 6° C., or may be about 5° C., while freezing temperatures may range from about −20° C. to about 0° C., or about −15° C. to about −5° C., or about −18° C. to about −8° C., or may be about −10° C. For example, a must chiller may be set to about 11° C. to about 13° C. Other methods utilise 3600 freezing or chilling and/or ACVCS next generation freezing technology. Once frozen or chilled, the skins can be stored under these conditions until required. Frozen grape skins can be thawed for drying or may be dried directly by freeze drying.

In certain circumstances, cryomaceration may be used as a chilling or freezing methodology. For example, following crushing or pressing, the grape skins may be exposed to low or cold temperatures for more than 1 hour. Exemplary temperatures include about −18° C. to about 0° C., or about −15° C. to about 0° C., or about −10° C. to about 0° C., or about −5° C. to about 0° C., or about 4° C. to about 15° C., about 5° C. to about 15° C., about 6° C. to about 12° C., about 8° C. to about 12° C., or about 10° C. The duration may be dependent on the temperature, for example, from about 2 hours up to 7-14 days. The cooling may utilise cryogens/coolants such as solid carbon dioxide (dry ice), liquid carbon dioxide, or nitrogen. Notably, the cold shock from cryomaceration produces a larger volume of water in the grape skins and the weight of the skins helps to break up the cellular membrane (cell cracking).

In a particular exemplification, grapes may be quickly frozen and then slowly thawed. For example, the grapes may be frozen to about −30° C. to about −10° C., or about −25° to about −15° C., or to about −20° C. (−4 F), using one or more cryogens, such as dry ice, and then thawed, for example, over about 18 hours, over about 20 hours, over about 24 hours, over about 36 hours, or over about 48 hours. In particular aspects, the process may be carried out so as to maximise ice crystals formation, to damage the grape skin tissue, and enhance levels of beneficial sulphur compounds such as volatile thiols and their precursors. It is also possible to utilise an extraction process, e.g., water extraction, for the thawed grape skins. See, for example, Jelley et al., 2016; Jelley et al., 2020. As one specific option, the grapes may be frozen, thawed, and then soaked in water. The water mixture may then be separated and further processed. For example, evaporation can be used to dry down the mixture (e.g., a rotary evaporator may be used). The water may also be treated by filtration prior to evaporation. For example, one or more of pad filters, mesh filters, centrifuge filters, or other brewing filters may be used. The dried down and/or filtered product (e.g., paste product) can be dried and milled as described below. Without wishing to be bound by theory, it is believed that the soaking process may assist with extraction of Cys-3SH and GSH-3SH thiol precursors from the grape skins.

It is expected that the grape skins will be substantially dried following chilling or freezing. Drying will include freeze drying and may also involve other methodology. For example, evaporation may be used in combination with freeze drying. Freeze drying techniques are widely known and commonly used. The freeze drying process may range from 5 to 60 hours; or 10 to 50 hours; or 15 to 40 hours; or about 20 to about 30 hours. Noted for freeze drying are ranges of about 23 to about 27 hours, or about 24 to about 26 hours. Importantly, freeze drying may be utilised to retain the beneficial compounds in the grape skins, for example, terpenes and key C6 compounds, including volatile thiols and their precursors, as described in detail herein.

It may be desirable to use a particular freeze drying process for obtaining the dried product. See, e.g., de Torres et al., 2015. For example, a drying program may be used as part of an automated drying system. The process may include multiple drying steps, e.g., with step wise increases and reductions in temperature. Preferably, a primary drying setting is used for sublimation, followed by one or more secondary drying settings that are used to remove residual moisture. In particular aspects, the top temperature of the process does not exceed 35° C., or alternatively, the top temperature does not exceed 50° C. In other aspects, the temperature of the drying process ranges between −10° C. to 70° C. In one other aspect, at least 24 hours of dying is utilised. As specific exemplifications, chamber batch drying or continuous tunnel drying may be used, for example.

In certain aspects, an additive may be used to accelerate or otherwise assist the drying process. For example, pea starch or other drying aids may be utilised. Also noted as a drying aid is dextrin and various forms of dextrin, for example, maltodextrin. In addition, the dried products may be made to be cold water soluble. The resulting dried product may then be milled into a flake or powder form. Exemplary powders may have a sieve size of about 1 mm or less. Milling methods are well known and widely used in the art. Standard mesh sizes may be used to produce the powder, for example, US 20, US 23, US 30, US 35, US 40, US 45, or US 50 mesh sizes may be used. The sieve size for the powder may range from 8 mm to 0.3 mm; or 4 mm to 0.4 mm; or 4 mm to 1 mm; or may be about 4.0 mm, about 3.15 mm, about 3 mm, about 2.8 mm, about 2 mm, about 1 mm, about 0.84 mm, about 0.71 mm, about 0.59 mm, about 0.5 mm, about 0.47 mm, about 0.465 mm, about 0.437 mm, about 0.4 mm, about 0.355 mm, or about 0.3 mm. Powder may be packaged into nitrogen flushed bags to protect against oxidation, for example, 0.5 kg to 30 kg bags, or 1 kg to 20 kg bags.

It is expected that the composition of this disclosure will contain thiol precursors in sufficient quantities so as to provide a readily utilised source of such compounds. For example, the composition may include C3MH, at a concentration of at least 0.5 μg per gram, at least 0.8 μg per gram, at least 1 μg per gram, at least 1.2 μg per gram, at least 1.5 μg per gram, at least 1.8 μg per gram, at least 2 μg per gram, at least 2.2 μg per gram, at least 2.5 μg per gram, at least 3 μg per gram, at least 3.2 μg per gram, or at least 3.5 μg per gram, or a liquid equivalent thereof. As further examples, the composition may include CG3MH at a concentration of at least 0.05 μg per gram, 0.08 μg per gram, at least 0.1 μg per gram, at least 0.12 μg per gram, at least 0.15 μg per gram, at least 0.18 μg per gram, at least 0.2 μg per gram, at least 0.22 μg per gram, or at least 0.25 μg per gram, or a liquid equivalent thereof. As further examples, the composition may include GC3MH at a concentration of at least 0.15 μg per gram, at least 0.2 μg per gram, at least 0.25 μg per gram, at least 0.3 μg per gram, at least 0.35 μg per gram, at least 0.4 μg per gram, at least 0.45 μg per gram, at least 0.5 μg per gram, at least 0.55 μg per gram, at least 0.6 μg per gram, or at least 0.65 μg per gram, or a liquid equivalent thereof. As further examples, the composition may include G3MH at a concentration of at least 1.8 μg per gram, at least 2 μg per gram, at least 2.5 μg per gram, at least 2.8 μg per gram, at least 3 μg per gram, at least 3.2 μg per gram, at least 3.8 μg per gram, at least 4 μg per gram, at least 4.2 μg per gram, or at least 4.5 μg per gram, or a liquid equivalent thereof.

It will be understood that the grape skin composition disclosed herein may be utilised on its own. In addition, as noted herein, the composition of the present disclosure may be combined with one or more hops or one or more components obtained from hops. As exemplifications, the composition that includes hops or hops component(s) may be formulated as a powder, pellet, or plug. Alternatively, the composition that includes hops or hops components may be liquid in form. In certain aspects, hops products may be prepared so as to be intermixed with grape skin concentrate. For example, grape skin concentrate powder may be added during preparation of hops pellets or hops plugs to produce a combined product, or grape skin concentrate powder may be added with hops powder to produce a combined product. In other aspects, hops or hops components may be modified with grape skin concentrate. For example, whole leaf hops, hops plugs, or hops/lupulin pellets (e.g., cryogenically prepared hops pellets) may be dipped, rolled in, or dusted with grape skin concentrate powder to produce a combined product.

Combination products include, for example, hybrid pellets having one or more hops or hops components and grape skin components. These may have desirable levels of aromatic compounds, and may also have synergistic properties in the fermentation. Various combination products are set out herein and are encompassed by this disclosure. As particular examples, grape skin concentrate composition obtained from New Zealand Sauvignon Blanc wine grapes may be pelletised with one or more American, German, or English hops or hops components, or alternatively, one or more Australian, New Zealand, or South African hops or hops components, or any combination of these. These exemplifications are provided as illustrations only, and other combinations are considered to be encompassed by this disclosure.

### Methods of Using Compositions Comprising Grape Skin Concentrates

The applicant has found that compositions comprising grape skin concentrates include chemical compounds that are useful for the preparation of fermented beverages such as beers, beer-wine hybrids, fruit beers, and ciders, as well as kombuchas. Particularly noted are the sulphur compounds such as volatile thiols, such as 3MH, 3MHA, and 4MMP, and their precursors. Therefore, it is expected that the beverages produced according to the present disclosure will include beneficial levels of sulphur compounds, including volatile thiols and their precursors.

As non-limiting exemplifications, the produced beverage may include at least 50 ng/L to at least 12,000 ng/L 3MHA; and/or at least 150 ng/L to at least 20,000 ng/L 3MH; and/or at least 5 ng/L to at least 100 ng/L 4MMP. For 3MH, in particular, levels may be at least 40 ng/L, at least 50 ng/L, at least 60 ng/L, at least 70 ng/L, at least 80 ng/L, at least 90 ng/L, at least 100 ng/L, at least 200 ng/L, at least 300 ng/L, at least 400 ng/L, at least 500 ng/L, at least 600 ng/L, at least 700 ng/L, at least 800 ng/L, at least 900 ng/L, at least 1000 ng/L, at least 1100 ng/L, at least 1200 ng/L, at least 1500 ng/L, at least 1700 ng/L, at least 1900 ng/L, at least 2100 ng/L, or at least 10,000 ng/L. For 4MMP, levels may be at least 0.5 ng/L, at least 1 ng/L, at least 1.2 ng/L, at least 1.4 ng/L, at least 1.6 ng/L, at least 1.8 n/L, at least 2 ng/L, at least 2.2 ng/L, at least 2.4 ng/L, at least 2.6 ng/L, at least 2.8 ng/L, at least 3 ng/L, at least 15 ng/L, or at least 150 ng/L, at least 175 ng/L, at least 200 ng/L, at least 225 ng/L, at least 250 ng/L, at least 300 ng/L, at least 325 ng/L, at least 330 ng/L, or at least 400 ng/L. For 3MHA, levels may be at least 2.5 ng/L, at least 5 ng/L, at least 6 ng/L, at least 7 ng/L, at least 8 ng/L, at least 9 ng/L, at least 10 ng/L, at least 11 ng/L, at least 12 ng/L, at least 15 ng/L, at least 20 ng/L, at least 30 ng/L, at least 35 ng/L, at least 40 ng/L, at least 45 ng/L, at least 55 ng/L, at least 60 ng/L, at least 65 ng/L, at least 70 ng/L, at least 75 ng/L, or at least 500 ng/L.

The produced beverage may also include one or more thiol precursors. For example, the beverage may include C3MH, at a concentration of at least 10 μg/L, at least 15 μg/L, at least 25 μg/L, at least 30 μg/L, at least 35 μg/L, at least 40 μg/L, at least 45 μg/L, at least 50 μg/L, at least 55 μg/L, or at least 60 μg/L, or a liquid equivalent thereof. As further examples, the beverage may include CG3MH at a concentration of at least 20 μg/L, 25 μg/L, at least 30 μg/L, at least 35 μg/L, at least 40 μg/L, at least 45 μg/L, at least 50 μg/L, at least 60 μg/L, at least 65 μg/L, at least 70 μg/L, or at least 75 μg/L, or a liquid equivalent thereof. As further examples, the composition may include GC3MH at a concentration of at least 8 μg/L, at least 10 μg/L, at least 15 μg/L, at least 20 μg/L, at least 25 μg/L, at least 30 μg/L, at least 35 μg/L, at least 40 μg/L, at least 45 μg/L, at least 50 μg/L, at least 55 μg/L, or at least 60 μg/L, or a liquid equivalent thereof. As further examples, the composition may include G3MH at a concentration of at least 70 μg/L, at least 75 μg/L, at least 80 μg/L, at least 85 μg/L, at least 90 μg/L, at least 95 μg/L, at least 100 μg/L, at least 105 μg/L, at least 110 μg/L, or at least 115 μg/L, or a liquid equivalent thereof.

The fermented beverage produced with the composition is expected to include increased levels of volatile thiols as compared to a beverage produced without the composition. For example, 3MH levels may be increased 1.5 fold to 15 fold, or at least 1.5 fold, at least 2 fold, at least 3 fold, at least 4 fold, at least 5 fold, at least 10 fold, at least 15 fold, or at least 20 fold. The 4MMP levels may be increased 2 fold to 20 fold, or at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 5 fold, at least 10 fold, at least 15 fold, at least 20 fold, or at least 25 fold. The 3MHA levels may be increased 1.2 fold to 12 fold, or at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 5 fold, at least 10 fold, at least 15 fold, or at least 20 fold.

For producing the beverages of the present disclosure, various organisms may be used. Brewer's yeasts that are useful for fermentation are particularly noted. Exemplary yeasts include Saccharomyces yeasts, for example, Saccharomyces cerevisiae. Other yeasts include but are not limited to Brettanomyces, Candida, Hanseniaspora, Kloeckera, and Pichia yeasts, for example: B. bruxellensis, B. custersii, B. lambicus, B. intermedius, B. anomalus, C. stellata, C. zemplinina, C. pulcherrima, H. uvarum, H. osmophila, H. guilliermondii, K. apiculata, K. javanica, K. cortices, P. kluyveri, and S. pastorianus, S. uvarum, amongst others. Included also are Dekkera anomala, Naumovozyma dairenensis, and Debaryomyces spp. yeasts.

Yeast strains that are used for volatile thiol production (e.g., yeast suitable for biotransformation of thiol precursors into volatile thiols) are particularly noted, for example, S. cerevisiae and P. kluyveri strains. For kombucha, both yeast and bacterial cultures may be used, e.g., yeast such as a Zygosaccharomyces, Candida, Torulaspora, Pichia, Brettanomyces/Dekkera, Schizosaccharomyces, and/or Saccharomyces strains (as examples, Saccharomyces cerevisiae or Zygosaccharomyces kombuchaensis) along with bacteria, such as Komagataeibacter and/or Gluconobacter strains (as examples, Komagataeibacter xylinus or Komagataeibacter kombuchae). Combinations of any of the above organisms, including specific combinations with S. cerevisiae may also be used.

Exemplary strains include effective thiol producing strains, for example, strains that are used to convert thiol precursors into volatile thiols (e.g., 3MH or 4MMP), or strains that are used to produce the volatile thiols from other compounds. Other strains may convert 3MH to 3MHA, or produce other chemical conversions in relation to volatile thiol molecules. For example, various Saccharomyces and/or Pichia strains may be used for volatile thiol production. It is also possible to include yeasts (e.g., inactive yeasts) that produce glutathione, as a protection from oxidation. Yeast with increased β-lyase activity (e.g., increased expression levels and/or increased enzymatic function) and/or increased β-glucosidase activity (e.g., increased expression levels and/or increase enzymatic function) are specifically noted. Such yeast may be genetically engineered, for example, to include one or more mutations or chromosomal insertions. In addition to or as an alternative to this, such yeast may include one or more expression constructs, for example, one or more plasmids to provide β-lyase and/or β-glucosidase expression.

Particular P. kluyveri strains include, but are not limited to: PK-KR1, PK-KR2, (see, e.g., US 20170183612), and DSM 33235. Pichia kluyveri var kluyveri is specifically noted. Noted also is the commercially available product FROOTZEN® (Chr Hansen), which comprises P. kluyveri isolated from New Zealand grape juice. Particular S. cerevisiae strains include, but are not limited to: L2056, NT116, VIN7, VIN13, VL3, X5, and QA23 strains. Commercially available strains may be obtained, for example, from Wyeast, White Labs, Omega Yeast, Fermentis, Escarpment Laboratories, and other suppliers. Particular strains of interest include Wyeast 1318 London Ale III™ yeast, as well as Wyeast 1056 American Ale yeast; and also WLP066 London Fog Ale yeast, WLP518 Opshaug Kveik Ale yeast, and WLP077 tropicale yeast blend from White Labs; along with SafAle™ S-33, SafAle™ K-97, and SafAle™ S-04 from Fermentis; and OYL-061 Voss Kveik, or OYL-402 or OYL-405 yeast from Omega Yeast. Also noted are Berkeley Labs Tropics thiol yeast, Lallemand Lalbrew yeast, and other yeast with increased levels of β-glucosidase or β-lyase activity. Any mixture of yeast strains can be used to maximise production and biotransformation levels.

Notably, it is possible to obtain substantially increased levels of volatile thiols in beverages prepared using the composition of this disclosure in combination with yeast strains used for converting thiol precursor compounds to volatile thiols. For example, as compared to beverages prepared without the composition and without such yeast, levels of 3MH may be increased at least 30 fold, at least 35 fold, at least 40 fold, at least 45 fold, at least 50 fold, at least 60 fold, at least 70 fold, at least 80 fold, at least 90 fold, at least 100 fold, at least 120 fold, at least 180 fold, or at least 200 fold.

Bacterial strains that are used for lactic acid production are particularly noted, for example, strains of the order Lactobacillales. For example, lactic acid producing strains (i.e., lactic acid bacteria) may be utilised. These include, but are not limited to Lactobacillus, Lactococcus, Pediococcus, and Streptococcus strains. Specifically noted for fermentation are Lactobacillus acidophilus, Lactobacillus brevis, Lactobacillus buchneri, Lactobacillus delbruckii, Lactobacillus lactis, Lactobacillus plantarum, and Lactobacillus rhamnosus strains. Also noted are Pediococcus acidilactici, Pediococcus damnosus Pediococcus parvulus, Pediococcus pentosaceus strains. Commercially available lactic acid bacteria may be obtained, for example, from Wyeast, White Labs, Omega, and other suppliers. Particular strains of interest include WLP677, WLP672 from White Labs; 5335, 5223-PC, 4335 from Wyeast; OYL-605 from Omega; MIP-911, MIP-912, MIP-913, MIP-914 dein Propagate Lab; RVA 600 from RVA Yeast Labs. Other strains of interest include OYL-606 from Omega; MIP-920 from Propagate Lab; RVA 601 from RVA Yeast Labs; WLP661 from White Labs; 5733 from Wyeast. Any mixture of bacterial strains can be used to maximise lactic acid production and activity levels. It will be understood that mixtures of bacteria and yeast strains may also be utilised.

When preparing a beverage, the composition of this disclosure may be introduced together with the hops or hops component(s). Alternatively, introduction of the composition and the hops/hops component(s) may be separate. Introduction of the composition may be at one or more points during the beverage processing. The introduction of the hops/hops component(s) may be at one or more points during the beverage processing. For preparing fermented beverages, particular ratio amounts of the grape skin composition and the hops may be used. As exemplifications, ratio amounts of about 2.5 to about 1; about 2 to about 1; about 1 to about 1; about 1 to about 0.75; about 1 to about 0.5; about 1 to about 0.25 of grape skin composition to hops may be employed.

In other aspects, a combination of grape skin composition and hops may comprise or may consist essentially of about 10% to about 90% grape skin composition (w/w) and about 90% to about 10% hops (w/w), or about 15% to about 85% grape skin composition (w/w) and about 85% to about 15% hops (w/w), or about 20% to about 80% grape skin composition (w/w) to about 80% to about 20% hops (w/w), or about 20% to about 40% grape skin composition (w/w) to about 80% to about 60% hops (w/w), or about 25% to about 35% grape skin composition (w/w) to about 75% to about 65% hops (w/w).

The grape skin composition may be added when fermentation is already in progress. For example, the grape skin composition may be added to a wort stream at about 4 degrees Plato to about 8 degrees Plato, or at about 5 degrees Plato to about 7 degrees Plato. Upon addition of the grape skin composition, fermentation can then be continued, e.g., at temperatures of about 18° C. to about 22° C., or about 19° C. to about 21° C. As one specific option, the grape skin composition may be added before the start of fermentation, for example, during the boiling phase or whirlpool phase for brewing. It is also possible to add the grape skin composition after fermentation or proximate to the end of fermentation. In the same way, it is possible to add hops or hops component(s) after fermentation or proximate to the end of fermentation.

It will be understood that various methods may be combined or modified for beverage preparation, and the composition of this disclosure is not limited to use with particular methodologies. For example, where brewing is carried out, it will be possible to use mash hopping, first wort hopping, whirlpool hopping (e.g., hop stand), dry hopping, wet hopping, hop bursting, hop addition during the boil, hop addition proximate the end of the boil, or any combination of these methods. For dry hopping, it will be possible to employ dry hop addition in the primary fermenter and/or dry hop addition in the secondary fermenter (e.g., loose or contained). Hop addition (hopping) may be by introduction of one or more hops or hops component(s), or by combinations of one or more hops and hops component(s).

As one exemplification, the grape skin composition may be added to the brewing process as a hot side addition. For example, the composition can be introduced into the mash tun and/or kettle/whirlpool. This would introduce concentrated thiol precursors, which could then be converted to volatile thiols during the fermentation stage. Specifically, this may be achieved by addition of a β-lyase enzyme and/or by fermentation with either a yeast strain used to boost volatile thiol compounds, for example, Cosmic Punch™ (OYL-402; Omega Yeast). Alternatively, or in addition to this, a standard strain of brewer's yeast could be used in combination with a co-fermentation strain, such as P. kluyveri. For example, the P. kluyveri could be added on day 1 of fermentation, and within the first 48 hours covert the thiol precursors into volatile thiol compounds. At this stage, a further standard strain of brewer's yeast could then be added to complete the fermentation. As a further option, dry hop additions may be made with further addition(s) of the grape skin composition to re-introduce thiol precursor, alongside the introduction of hops or hops component(s) (e.g., lupulin, terpenes, and/or esters) for combined fermentation effects.

In certain circumstances, it may be desirable to enrich the levels of beneficial sulphur compounds that are available during fermentation. In particular, purified sulphur compounds may be introduced prior to or during fermentation, including purified volatile thiols such as one or more of 3MH, 3MHA, and 4MMP, and/or their precursors. Purified compounds may also be introduced after fermentation, to supplement the levels of beneficial sulphur-containing components in the final beverage. It will be understood that the levels of sulphur compounds, for example, volatile thiols and their precursors, as well as phenolics, antioxidants, carbohydrates, etc, may be monitored before, during, and/or after fermentation. Various measurement methodologies can be used, as described here.

To maintain maximal levels of sulphur compounds, including the levels of volatile thiol compounds and their precursors, it is possible to use fining agents to negate metal catalysts and residual polyphenols during fermentation. Exemplary agents are noted herein. It is also possible to use de-oxygenation and inert gas purging during fermentation. For example, carbon dioxide and/or nitrogen gas may be introduced. In addition, it is possible to include one or more antioxidants/preservatives before, during, or after fermentation. Non-limiting examples of these compounds are provided herein.

One or more enzymes may also be used to optimise pressing and maximise aromatic compounds. For example, one or more pectolytic enzymes may be used. This includes commercially available enzymes such as those of LAFAZYM® PRESS. Also noted are aroma enhancing enzymes such as Rapidase® Expression Aroma enzymes and Lallemand Aromazyme™ enzymes. Noted as well are various lyase products, e.g., sulphur lyase such as Endozym® Thiol. Furthermore, it is possible to use higher fermentation temperatures (irrespective of yeast strain(s) used) to increase the release volatile thiols by yeast strains. For example, temperatures of at least 17° C., at least 18° C., at least 19° C., at least 20° C., at least 23° C., at least 25° C., at least 27° C., at least 30° C., at least 33° C., at least 35° C., or at least 37° C. can be used, for at least part of the fermentation period. In particular, Kveik yeast fermentations can be carried out from about 30 to about 37° C. It is also possible to reduce temperatures for the later stages of fermentation (e.g., reduction to about 15° C. to about 16° C.) to preserve the volatile compounds that have been released.

Following fermentation, the beverage will be ideally chilled and stored at low temperatures to minimise oxidation reactions, and also to reduce hydrolysis reactions converting 3MHA TO 3MH. It would therefore be preferable to keep the beverage at a temperature as low as possible without freezing during the time periods following fermentation, prior to bottling (or canning or kegging), and prior to consumption. Exemplary temperatures include but are not limited to temperature of less than 16° C., less than 15° C., less than 14° C., less than 12° C., less than 10° C., less than 8° C., less than 6° C., or less than 4° C. It is also preferably to carry out bottling, canning, or kegging as quickly as possible following fermentation, to reduce oxygen exposure.

It will be understood that the compositions of the present disclosure are useful for the preparation of various fermented beverages. Included amongst these are beers, such as beers made from cereals (e.g., barley malt), maize, millet, oats, rice, rye, sorghum, or wheat, or any combination of these. Specifically included are ales such as amber ales, Belgian style ales, blonde ales, brown ales, sour ales, wild ales, and also pale ales, such as American Pale Ale, India Pale Ale, and New Zealand Pale Ale; bocks, such as German bocks; lagers, such as pale lagers and dark lagers, as well as Vienna lagers; pilsners; porters, such as Baltic porters; radlers; saisons; session beers; and stouts, including Irish stouts and Imperial stouts. Particular ales of interest include Hazy IPAs, New England IPAs, Double IPAs, Triple IPAs, Sour IPAs, Fruit IPAs, Oat Cream IPAs, Milkshake IPAs, and Tropical IPAs. Other beers include weissbier, Berliner weisse, witbier, maibocks, English bitter, Biere de Garde, marzen, dunkel, dunkelweizen, and doppelbock beers.

Included also are various beer-wine hybrids, for example, beers brewed in combination with one or more types of wine grapes. White wine grapes are noted, in particular, for beer-wine hybrids. For example, Sauvignon Blanc grapes may be used or any other white wine grape, including those described herein, and any combinations of these. Also included are various ciders, such as dry ciders, and specifically including cloudy and clear ciders, and single and multiple fruit ciders, for example, ciders comprising juice from one or more of: crab apples, apples, pears, kiwifruits, and berries, such as strawberries, blackberries, raspberries.

Various fruit beers are further included, such as beers brewed with one or more fruits, for example, guavas, mangos, papayas, passionfruit, pineapple, plums, peaches, apricots, citrus, cherries, and berries, including Boysenberries, blueberries, raspberries, strawberries, and any combination of the preceding. Fruit may be introduced during beermaking via whole fruits, fruit parts, fruit juice, fruit purees, fruit extracts, or other means, and may act as an adjunct or flavouring for the beer. Kombuchas are included as well, e.g., kombuchas flavoured with one or more fruit juices and/or one or more spices. It will be understood that these are exemplifications, only, and other types of beers, beer-wine hybrids, fruit beers, ciders, and kombuchas will be known and well within the purview of the skilled person.

In various aspects, the composition of this disclosure may be utilised at appropriate levels to achieve the desired levels of thiol precursors and volatile thiol compounds. As exemplifications, the composition may be introduced at amounts of at least 2 g/L, or at least 3 g/L or at least 4 g/L, or at least 5 g/L, or at least 6 g/L, or at least 7 g/L, or at least 8 g/L, or at least 9 g/L, or at least 10 g/L, or ranging at about 2 g/L to about 8 g/L, or about 3 g/L to about 8 g/L, or about 4 g/L to about 8 g/L, or about 5 g/L to about 8 g/L, or about 6 g/L to about 8 g/L. As further exemplifications, the composition may be introduced at amounts of at least 2 ml/L, or at least 3 ml/L or at least 4 ml/L, or at least 5 ml/L, or at least 6 ml/L, or at least 7 ml/L, or at least 8 ml/L, or at least 9 ml/L, or at least 10 ml/L, or ranging at about 2 ml/L to about 8 ml/L, or about 3 ml/L to about 8 ml/L, or about 4 ml/L to about 8 ml/L, or about 5 ml/L to about 8 ml/L, or about 6 ml/L to about 8 ml/L.

It will be understood that the amounts of the composition added may be adjusted as desired. For example, the amounts used may vary depending on the other ingredients being utilised (e.g., yeast strains, bacterial strains, enzymes, hops or hops component(s), etc), or depending on whether the composition has been formulated to include one or more additional ingredients (e.g., one or more yeast strains, bacterial strains, enzymes, hops or hops component(s), etc). Amounts added may also be adjusted depending on the concentration of thiol precursors in the composition.

When preparing fermented beverages, it is expected that addition of the composition prior to fermentation will increase thiol precursors levels as compared to pre-fermentation mixtures prepared without the composition. For example, the levels of C3MH may increase 2 fold to 9 fold, or at least 1.5 fold, at least 2 fold, at least 3 fold, at least 4 fold, at least 5 fold, at least 6 fold, at least 7 fold, at least 8 fold, at least 9 fold, at least 10 fold, at least 15 fold, or at least 20 fold. The levels of CG3MH may increase 1.05 fold to 1.2 fold, or at least 1.05 fold, at least 1.1 fold, at least 1.5 fold, or at least 2 fold. The levels of GC3MH may increase 1.1 fold to 1.4 fold, or at least 1.05 fold, at least 1.1 fold, at least 1.2 fold, at least 1.3 fold, at least 1.4 fold, at least 1.5 fold, at least 2 fold, or at least 3 fold. The levels of G3MH may increase 1.2 fold to 1.8 fold, or at least 1.2 fold, at least 1.3 fold, at least 1.4 fold, at least 1.5 fold, at least 1.6 fold, at least 1.7 fold, at least 1.8 fold, at least 2 fold, or at least 3 fold. In particular, when preparing brewed beverages, such increases in thiol precursors may be seen in the wort.

Therefore, with the grape skin concentrate provided as part of a beneficial composition (e.g., powder, flakes, etc), this provides an ideal ingredient for use in the fermentation process. In particular, the composition of the present disclosure provides a stable source of volatile thiols and their precursors, which can be introduced into the fermentation process to increase highly desirable aromatic compounds (e.g., tropical, fruity, etc) in a finished beverage. The production of beers, beer-wine hybrids, fruit beers, and ciders is specifically envisioned, along with production of kombuchas. For example, to brew highly aromatic beer, it is possible to use the grape skin concentrate composition as a replacement for hops, i.e., fermentation without the addition of hops. Alternatively, at least one hops in combination with at least one grape skin concentrate composition may be introduced to the active fermentation. In various alternatives, the hops may be combined with the grape skin extract composition before or upon introduction to fermentation mixture, and the hops may be provided in whole leaf form or as hops component(s), e.g., hops powder, hops extract (e.g., lupulin, hops terpenes, and/or hops esters), hops pellet, or hops plug. In one particular aspect, the introduction may be carried out as part of a dry hops addition. Notably, the combination of hops and grape skin extract being introduced into the active fermentation allows for biotransformation to produce aromas and flavours.

Accordingly, in various aspects, this disclosure encompasses combinations which include the grape skin composition and at least one additional ingredient. This includes, for example, one or more hops, one or more hops component(s) (e.g., lupulin, hops terpenes, and/or hops esters), one or more yeast, one or more enzymes, one or more fruit concentrates (e.g., juice, flesh, or whole fruit, or combinations of these), one or more sulphur compounds (e.g., any thiol precursor or combinations of these), or other ingredients as set out herein, or any combination of the preceding. In certain embodiments, the grape skin composition may be provided in liquid or semi-liquid form (e.g., mixed in water or other solvent(s)). In certain embodiments, the grape skin composition may be provided as a dry form (e.g., powder). In further embodiments, the one or more additional ingredients may be provided in liquid, semi-liquid (e.g., paste, gel), or dry (e.g., powder) form to produce the combination.

Particularly encompassed are combinations comprising the grape skin composition and at least one yeast. As an example, the grape skin composition may be combined with one or more yeast strains used for volatile thiol production, as described in detail herein. Also encompassed are combinations comprising the grape skin composition and one or more hops and/or hops component(s). As an example, the grape skin composition may be combined with one or more hops/hops component(s) used for volatile thiol production, as described in detail herein. Further encompassed are combinations comprising the grape skin composition and one or more hops or hops component(s) and also one or more yeast. For example, the grape skin composition may be combined with one or more hops or hops component(s) used for volatile thiol production and also one or more yeast used for volatile thiol production. Other combinations may be obtained and used in the methods set out herein.

It is envisioned that the composition of this disclosure may be included in kit, for example, a kit for preparing a beverage, such as a fermented beverage. The composition may be provided in one or more containers in the kit. Additional components may also be provided with the kit, for example, one or more hops or hops component(s), or one or more yeast, one or more bacteria, one or more enzymes, one or more sulphur compounds, one or more fruit concentrates, or any combination thereof. The composition may be provided as a combined formulation as described in detail herein. Optionally, instructions may be provided with the kit, as well as any other item, such as any number of containers, labels, and/or tools, including bottles, injectors, scoops, etc. The instructions for the kit may include information as to storage, usage, or testing, amongst other information. The kit may optionally include one or more reagents for testing thiol precursor levels or volatile thiol levels.

### EXAMPLES

The examples described herein are provided for the purpose of illustrating specific embodiments and are not intended to limit this disclosure in any way.

### Example 1: Process for Producing Grape Skin Compositions

### Materials and Methodology

High thiol content grapes were sourced from Nautilus Estate in Marlborough, New Zealand. Predominately Sauvignon blanc MS clone (UCD Clone 1) were used, alongside Bordeaux clones (Clone 316 and 317). Blocks noted for their historically high thiol content were selected, and harvested using a Gregoire and Pellenc machine destemming harvesters. The grapes were harvested between 21.5 and 22 degrees Brix. During the harvesting, potassium metabisulphite was dosed at 66 grams per tonne and added to the destemmed grapes. The grapes were put through a crushing machine with 30 parts per tonne ascorbic acid and 20 ml per tonne of enzymes added (pectolytic enzymes). The crushed grape must was then transferred through a must chiller set to 12° C. and into a holding tank for 1 to 8 hours.

A 150 HL Diemme open screen press was used to extract and separate the juice from the skins. The press operated at a maximum pressure of 2 bar over a 150 to 180 minute long press cycle. The press operated at an ambient temperature with the grapes added at 12° C. and remaining at a similar temperature throughout pressing. Three lots of skins were carefully collected and frozen immediately post press. One at 720 litres per tonne, one at 750 litres per tonne, and the other after a full press cycle as straight ‘marc’. The skins were collected immediately from the press into plastic lined bins and given an additional dose of potassium metabisulphite and ascorbic acid (same dosages of each, as noted above). The bins of skins were kept in a cold room at 0° C. until collected in a refrigerated truck and transferred to a freezer facility where by they were frozen at −20° C.

The frozen skins were thawed gently over a 24 hour period at ambient temperatures, until in a free form, pliable state, then freeze dried over a period of 24-26 hours, with a maximum temperature of 50° C. held at the end of the run for 3-4 hours. The powder was dried to an average moisture content of 12%, with the yield of raw material in to finished powder averaging 34%. The resulting product was milled into a powder through a 3 mm mesh. The powder was bagged in nitrogen flushed mylar bags containing 1 kg, 10 kgs, or 20 kgs amounts.

### Results

The finished powder was pale green in colour, smooth, with a texture like fine grain or sand. The finished powder was analysed and found to be free of pathogen such as E. coli, Listeria, Salmonella and faecal coliforms. The powder contained total sugars between 30.9 g/100 g and 38.8 g/100 g. This included essentially equal portions of fructose and glucose.

### Example 1A: Extraction Process

In an alternative finishing process, the thawed grape skins (see Example 1) were added to a volume of pure water between 2 to 6 times the volume of grape skins. The mixture was stirred at room temperature for 1 hour and left to settle for 10 minutes. After that, the liquid was filtered from the solids by use of a brewing filter. The liquid extract was then dried down in vacuo. The resulting product resembled a dark green paste. The paste was then freeze dried and milled into a powder as in Example 1. The concentrations of thiol precursors in the powder from this method were: Cys-3SH—1.19 mg/kg and GSH-3SH—3.76 mg/kg.

### Example 2: Products Prepared Using Grape Skin Compositions

### Materials and Methodology

Brewing trials were carried out to assess the impact of the grape skin compositions when used as dry hop additions. The grape skin compositions of Example 1 were utilised as follows. A standard wort stream, comprised of malted barley and oats, was produced at a starting gravity of 1.050, or 12.5 degrees Plato, with bittering additions introduced at the start of the boil of Warrior hop extract. During the later boil and whirlpool, further additions of hops were made in restrained amounts. The resulting wort was cooled to 20° C., aerated and transferred into three 50 L temperature controlled, conical fermenters (FV1, FV2, FV3). Brewer's yeast (London Ale III™) was pitched, and the fermentation was started. When the fermentation was at 6 degrees Plato, additions were made to the different fermenters comprising of:
FV1—1 kg of ‘720L/T grape skin powder’ and 750 g of Nelson Sauvin hop pellets;
FV2—1 kg of ‘750L/T grape skin powder’ and 750 g of Nelson Sauvin hop pellets; and
FV3—750 g of Nelson Sauvin hop pellets.

The trials were allowed to continue fermenting in the presence of the grape skin powder and hop pellets for 48 hours, with fermentation capped at 21° C., and then these were dumped off the bottom of the tank. The beers were chilled, settled, and racked into kegs, and then packaged into 950 ml cans for storage.

### Results

The aim of this trial was to assess the differences between fermented beverages prepared with the grape skin powders added versus a control fermentation beverage with just hops, all from a single stream of wort. Surprisingly, a strong hazing effect was observed for the resulting beer prepared with the grape skin powders. This was deemed to be advantageous as there is a preference for hazy appearance beverages. It is highly beneficial, then, that utilisation of the grape skin compositions can be used to create a stable haze in finished beer.

A sensory panel was assembled to include qualified brewers trained to identify and evaluate beer samples. The following results were obtained: FV1 was found to have intense passionfruit aroma, with a white wine juicy flavour and soft mouth feel. FV2 was found to have strong tropical fruit aroma, overripe fruit flavour, and perceived ‘dankness’ with slightly higher bitterness levels. FV3 was found to have a subtle citrus aroma with underlying bitterness. The sensory panel found samples FV1 and FV2 to be ranked higher in preference and beer quality rating than FV3.

### Example 3: Preparation and Analysis of Composition

### Materials and Methodology

Grape skin compositions were prepared according to Example 1 for batches of Sauvignon blanc grapes (Marlborough, New Zealand). Presses were performed at 750 litres per tonne. Each composition was analysed by UPLC-MS/MS using a stable isotope dilution assay (Nyseos Laboratories, France).

### Results

Results for the composition are shown in Table 4. The results demonstrate that the different preparative methods produced compositions with similar levels for C3MH, CG3MH, GC3MH, and G3MH (Table 4). Certain batches of grapes resulted in compositions with higher levels of thiol precursors. This was due to the time of harvest, and the historical levels of thiols found in the individual blocks of grapes. Accordingly, it may be useful to estimate thiol precursor levels in the grapes in advance of processing, and target specific growing blocks and/or harvest windows.

### Example 4: Beverage Preparative Methods Using Thiol Boosting Yeast

### Materials and Methodology

In additional experiments, grape skin composition was prepared as noted (Example 1, using presses at 720 or 750 litres per tonne) and then used to produce beer. For lots 750-00 and 750-01 (750 L/t pressed), 720-0 and 720-02 (720 L/t pressed), the following methods were used.

(1) Lot 750-00: 5,500 kg of Marlborough Sauvignon Blanc marc was collected in March 2021 (autumn harvest). This was pressed at 750 L/t and processed into powder according to Example 1. A total weight of 4,032 kg pressed material was processed into 1,340.80 kg grape skin composition and packed into 10 kg foil bags. Also referred to as lot 7500 or 21-750-00. (2) Lot 750-01: 5,810.50 kg Marlborough Sauvignon Blanc marc was collected in March 2021 (autumn harvest). This was pressed at 750 L/t and processed into a powder according to Example 1. A total weight of 5,040 kg pressed material was processed into 1,644 kg grape skin composition and packed into 10 kg foil bags. Also referred to as lot 7501 or 21-750-01. (3)/(4) Lots 720-00 and 720-02: Marlborough Sauvignon Blanc marc was collected in 2020 autumn harvest. This was pressed at 720 L/t. This was processed into a powder according to Example 1 and packed into 15 kg foil bags. Also referred to as 7200 and 72002

For brewing, the experiments employed 100% barley malt fermentations with a target original gravity 15° P. The grape skin composition (powder form) was added to the whirlpool at 2 lb/bbl (˜7 g/L). The powder was added at temperature >180° F. The gravity increase was only 0.2 degree plato (from 14.7 to 14.9). Rest was 15 minutes. As a control, parallel experiments were performed in the absence of the grape skin composition. The malt bill was 100% barley and 14.9 SG. The wort was transferred and cooled to 70° F. Cosmic Punch™ yeast (Omega Yeast; OYL 402) was added at 10 million cells/ml/° P (15 million cells/ml). The fermentation proceeded in 300 ml flask volumes (air lock/anaerobic) for 2 weeks. Fermentation volumes were 300 ml and were anaerobic with air locks at 70-72° F. After 14 days of fermentation, samples were transferred off of the yeast slurry to assess. Samples were decanted and stored at −80° C. in 50 ml conical tubes with the addition of sodium metabisulfite, to assist in preventing oxidation of thiols. In parallel, 50 ml was frozen with 15 ppm sodium metabisulfite. Where wort precursor levels were measured, samples were frozen pre-fermentation after the whirlpool step. Samples were sent for UPLC-MS/MS analysis, as above.

### Results

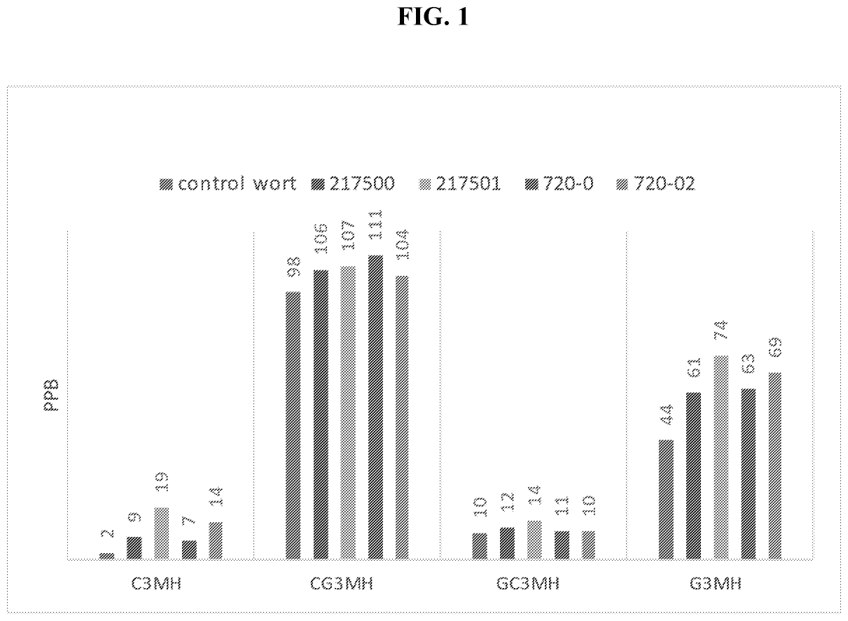

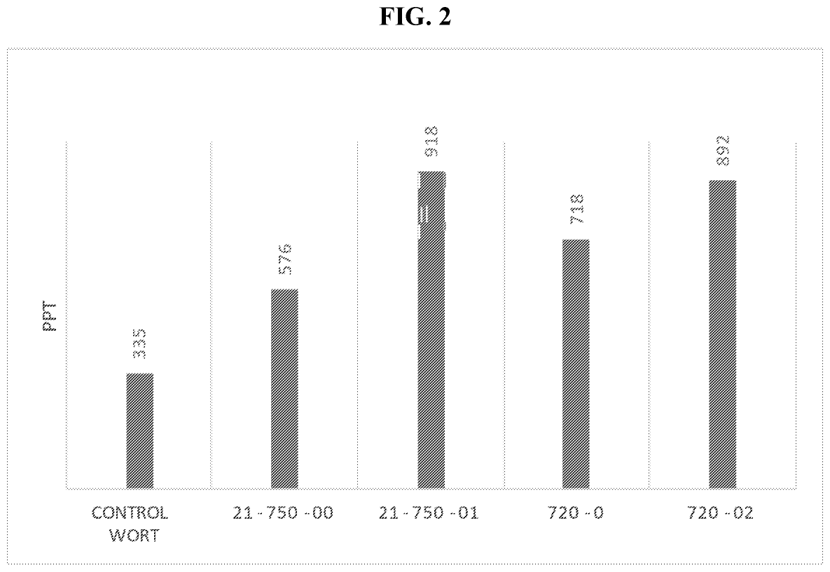

Results for the brewing process are shown in Tables 5-7. The results showed that the grape skin compositions produced significant increases in precursor thiol compounds in the wort, including increased levels of C3MH, CG3MH, GC3MH, and G3MH (Tables 5 and 6, and FIG. 1). The grape skin compositions further produced significant increases in volatile thiol compounds in the resulting beer, including increased levels of 3MH and 4MMP. Grape skin compositions also produced significant increases in 3MHA (Tables 5 and 7, and FIG. 2). At a trained sensory panel, the levels of thiols were noted as being clearly identifiable, and brought desirable aroma of tropical fruit, passionfruit and grapefruit to the beer. During sensory testing, the 750-01 and 720-02 were found to be the most thiol enriched. As before, differences in lots were attributed to the time of harvest, and the historical levels of thiols found in the individual blocks of grapes.

### Example 5: Beverage Preparative Methods Using Mash Hopping and Dry Hopping

In additional experiments, grape skin composition was prepared as noted (Example 1, pressing 750 litres per tonne) and then used to produce beer.

For lot 211165, the following methods were used. Grape skin compositions were prepared according to Example 1 for batches of Sauvignon blanc grapes (Marlborough, New Zealand). Presses were performed at 750 litres per tonne.

For brewing (mash hopping), a grain bill of 2-Row, Pilsner, Rye and Chit Malt was prepared and mashed in at 69° C. to create wort. During the whirlpool stage, 8 g/L grape skin composition (powder) was added to the wort at 82° C. Additionally, 16.5 ml INCOGNITO Mosaic™ (John I. Haas/HAAS; lupulin liquid/hop extract) was added as the wort was cooled to 70° F. and transferred to a fermenter. Cosmic Punch™ yeast (Omega Yeast; OYL 402) was added at 10 million cells/ml/° P (15 million cells/ml). Fermentation was set at 19° C., ramping up to 23° C. after 7 days. Alternatively, brewing employed dry-hopping with HBC 369 lupulin powder, which was added after fermentation at 13° C. As a control, parallel experiments were performed in the absence of the grape skin composition. The samples were analysed by UPLC-MS/MS, as above.

### Results

Results for the brewing process are shown in Table 8. The results show that free 3MHA levels were significantly elevated upon addition of the grape skin composition. At 8 g/L dose in the whirlpool, 3MHA levels were tripled. The beer produced was described by the brewery as having intense passionfruit aroma. The detectable threshold for 3MHA is about 50 to about 60 ng/L. Unexpectedly, the addition of dry hops during brewing masked the perception of that 3MHA and reduced free thiol levels in the final product. In particular, there was a reduction of approximately 12% in 3MH after dry hopping, and a reduction of approximately 45% in 3MHA after dry hopping. Conversely, 4MMP levels increased significantly upon dry hopping.

Accordingly, the disclosed methods may be utilised for enhancing or depressing thiol levels from fermentation, as needed. For example, to promote higher volatile thiol levels (e.g., 3MH and/or 3MHA), dry hops may be omitted or introduced in reduced quantities. To promote more of a balance between increased volatile thiols and hop character, the grape skin composition and hops can be introduced in conjunction, for example, as a combined product, and/or via co-administration, such as with sequential or simultaneous addition.

### Example 6: Beverage Preparative Methods Using Thiol Boosting Yeast

Brewing was carried out as described in Example 4. As controls: (1) brewing was performed in parallel without grape skin composition (powder); and (2) brewing was performed in parallel with control yeast (Omega Yeast; OYL-011; British Ale V). Samples were collected and assayed by UPLC-MS/MS.

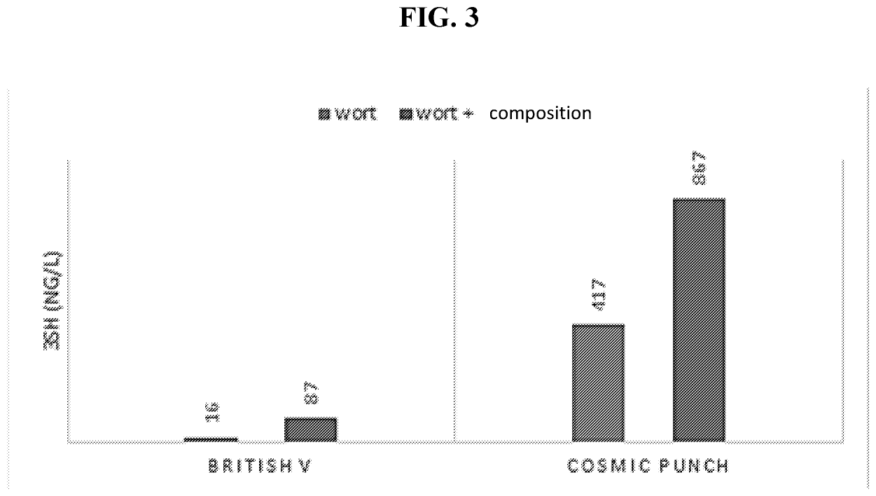

Results are shown in FIG. 3. 3MH levels were approximately 10 times higher when the grape skin composition was used in combination with Cosmic Punch™ yeast (Omega Yeast; OYL 402), as compared to control yeast (Omega Yeast; OYL 011; British Ale V).

TABLE 4
Thiol precursors in grape skin compositions

| | | | | cysteine- | cysteinyl- | γ-glutamyl- | glutathione- | cysteine- | glutathione- |
|---|---|---|---|---|---|---|---|---|---|
| | | | | 3MH | glycine-3MH | cysteine-3MH | 3MH | 4MMP | 4MMP |
| | | | | C3MH | CG3MH | GC3MH | G3MH | C4MMP | G4MMP |
| Sample | Material tested | Colour | Quantity | (μg/g) | (μg/g) | (μg/g) | (μg/g) | (μg/g) | (μg/g) |
| 217500 | Grape skin composition | Chestnut | 200 g | 1.2 | 0.1 | 0.3 | 2.8 | nd | nd |
| 217501 | Grape skin composition | Chestnut | 200 g | 3.0 | 0.2 | 0.6 | 4.0 | 0.0 | nd |

TABLE 5
Volatile thiols and thiol precursors in beer and wort samples

| | | | | 4-methyl- | | 3-sulfanyl- | 3- |
|---|---|---|---|---|---|---|---|
| | | | | 4-sulfanyl- | 3- | 4-meth- | sulfanyl- |
| | | | | pentan- | sulfanyl- | ylpentan- | hexan-1- |
| | | | | 2-one | hexan-1-ol | 1-ol | ol Acetate |
| | | | | 4MSP | 3SH | 3S4MPol | 3SHA |
| | | Material | Volume/ | (4MMP) | (3MH) | (3M4MP) | (3MHA) |
| Sample | Treatment | tested | Mass | ng/L | ng/L | ng/L | ng/L |
| 211175 | 33/34 Control Wort | Wort | 45 mL | | | | |
| 211175 | 34 Ph 7500 Wort | Wort | 45 mL | | | | |
| 211175 | 34 Ph 7501 Wort | Wort | 45 mL | | | | |
| 211175 | 34 Ph 7200 Wort | Wort | 45 mL | | | | |
| 211175 | 34 Ph 72002 Wort | Wort | 45 mL | | | | |
| 211175 | 33/34-1 Control | Beer | 45 mL | 0.7 | 335 | nd | 7.5 |
| 211175 | 34-2 7500 | Beer | 45 mL | 0.7 | 576 | nd | 10.2 |
| 211175 | 34-3 7501 | Beer | 45 mL | 2.1 | 918 | nd | 9.1 |
| 211175 | 34-4 7200 | Beer | 45 mL | 1.4 | 718 | nd | 7.0 |
| 211175 | 34-5 72002 | Beer | 45 mL | 1.7 | 892 | nd | 10.7 |

| | | cyste- | γ-glutamyl- | | | |
|---|---|---|---|---|---|---|
| | cysteine- | inylglycine- | cysteine- | glutathione- | cysteine- | glutathione- |
| | 3SH | 3SH | 3SH | 3SH | 4SMP | 4SMP |
| | C3MH | CG3MH | GC3MH | G3MH | C4MMP | G4MMP |
| Sample | μg/L | μg/L | μg/L | μg/L | μg/L | μg/L |
| 211175 | 2 | 98 | 10 | 44 | nd | nd |
| 211175 | 9 | 106 | 12 | 61 | nd | nd |
| 211175 | 19 | 107 | 14 | 74 | nd | nd |
| 211175 | 7 | 111 | 11 | 63 | nd | nd |
| 211175 | 14 | 104 | 10 | 69 | nd | nd |
| 211175 | | | | | | |
| 211175 | | | | | | |
| 211175 | | | | | | |
| 211175 | | | | | | |
| 211175 | | | | | | |

TABLE 6
Thiol precursors in wort samples

| Sample | C3MH μg/L | CG3MH μg/L | GC3MH μg/L | G3MH μg/L |
|---|---|---|---|---|
| control | 2 | 98 | 10 | 44 |
| wort | | | | |
| 750-00 | 9 | 106 | 12 | 61 |
| 750-01 | 19 | 107 | 14 | 74 |
| 720-0 | 7 | 111 | 11 | 63 |
| 720-02 | 14 | 104 | 10 | 69 |

TABLE 7
Volatile thiols in beer samples

| | 3SH ng/L | 4MSP ng/L | 3SHA ng/L |
|---|---|---|---|
| Sample | (3MH) | (4MMP) | (3MHA) |
| control | 335 | 0.7 | 7.5 |
| 750-00 | 576 | 0.7 | 10.2 |
| 750-01 | 918 | 2.1 | 9.1 |
| 720-0 | 718 | 1.4 | 7.0 |
| 720-02 | 892 | 1.7 | 10.7 |

TABLE 8
Volatile thiols in beer samples

| | | | | | 3-sulfanyl-4- | 3-sulfanyl- | 4-methyl-4- |
|---|---|---|---|---|---|---|---|
| | | | | 3-sulfanylhexan- | methylpentan- | hexan-1-ol | sulfanylpentan- |
| | | | | 1-ol | 1-ol | acetate | 2-one |
| | | | | 3SH | 3S4MPol | 3SHA | 4MSP |
| | | Material | Volume/ | (3MH) | (3M4MP) | (3MHA) | (4MMP) |
| Sample | Treatment | tested | Mass | ng/L | ng/L | ng/L | ng/L |
| 211165 | No grape skin | Beer | 50 mL | 1687 | 532 | 33 | 46 |
| | composition | | | | | | |
| 211165 | Grape skin | Beer | 50 mL | 1669 | 281 | 108 | 44 |
| | composition | | | | | | |
| 211165 | Grape skin | Beer | 50 mL | 1442 | 526 | 61 | 132 |
| | composition - Dry-hop | | | | | | |

### Example 6A: Beverage Preparative Methods Using Thiol Boosting Yeast

The grape skin composition was obtained as described in Example 1. Different lots of the grape skin composition were tested in these experiments. Beverage preparation methods were employed as described in Example 6, except where noted.

The control utilised yeast strain OYL-402 with no grape skin composition added. For the test samples, the grape skin composition was added to separate vats of hot wort. A standardised addition rate of 8 g/L or 16 g/L was used.

The results are shown in Tables 11-13, below. Beer produced using the grape skin compositions showed significantly higher levels of 3MH and positive sensory data, including increased haziness and fruity notes, including tropical, and melon notes.

In particular, the grape skin composition additions were observed to impart a more intense aroma, with relatively high levels of colloidal haze. Without wishing to be bound by theory, it was proposed that higher polyphenol levels could contribute to haze formation. In this way, it could be possible to employ higher or lower levels of the grape skin composition to increase or decrease haze formation, respectively.

### Example 7: Beverage Preparative Methods with Lupulin Powder

Grape skin composition was prepared as noted (Example 1, pressing 750 litres per tonne; composition lot 21-750-04) and then utilised in a beer brewing method. Also utilised was: Yakima Chief Hops (YCH) cryogenically prepared hops, which contained a blend of equal amounts of Citra, Mosaic, and Simcoe Cryo Hops®. The cryogenically prepared hops include a concentrated source of lupulin, as noted herein.

Additions were made at the whirlpool stage to achieve a total of 2 lb per bbl. This included addition of hops alone (˜8 g/L), grape skin composition alone (˜8 g/L), and mixtures of hops and grape skin composition. Accordingly, the following parameters were utilised: (1) 22022=0% grape skin composition, 100% hops (w/w); (2) 22023=100% grape skin composition, 0% hops (w/w); (3) 22024=20% grape skin composition and 80% hops (w/w; whirlpool addition); (4) 22025=40% grape skin composition and 60% hops (w/w; whirlpool addition).

The details for the whirlpool additions are shown below. For these tables, the alpha percentage indicates the amount of alpha acid as a percentage of total weight of the hops, while the IBU indicates the score on the International Bitterness Units scale. WP indicates whirlpool stage.

| | | | |
|---|---|---|---|
| Alpha % | Mass (g) | Added | IBU |

0% grape skin composition 100% hops

| | | | | |
|---|---|---|---|---|
| Citra Cryo Hops ® | 25.4% | 300 | WP | 30.5 |
| Simcoe Cryo Hops ® | 22.0% | 300 | WP | 25.4 |
| Mosaic Cryo Hops ® | 23.3% | 300 | WP | 28.0 |
| Grape skin composition | 0% | 0 | — | |

100% grape skin composition 0% hops

| | | | | |
|---|---|---|---|---|
| Citra Cryo Hops ® | 25.4% | 0 | — | 30.5 |
| Simcoe Cryo Hops ® | 22.0% | 0 | — | 25.4 |
| Mosaic Cryo Hops ® | 23.3% | 0 | — | 28.0 |
| Grape skin composition | 0% | 900 | WP | 0 |

20% grape skin composition 80% hops

| | | | | |
|---|---|---|---|---|
| Citra Cryo Hops ® | 25.4% | 240 | WP | 24.4 |
| Simcoe Cryo Hops ® | 22.0% | 240 | WP | 21.1 |
| Mosaic Cryo Hops ® | 23.3% | 240 | WP | 22.4 |
| Grape skin composition | 0% | 180 | WP | 0 |

40% grape skin composition 60% hops

| | | | | |
|---|---|---|---|---|
| Citra Cryo Hops ® | 25.4% | 180 | WP | 18.3 |
| Simcoe Cryo Hops ® | 22.0% | 180 | WP | 15.8 |
| Mosaic Cryo Hops ® | 23.3% | 180 | WP | 16.8 |
| Grape skin composition | 0% | 360 | WP | 0 |

The additions were made at temperature >180° F. The gravity increase was only 0.2 degree plato (from 14.7 to 14.9). Rest was 15 minutes. The malt bill was 100% barley and 14.9 SG. The wort was transferred and cooled to 70° F. Cosmic Punch™ yeast (Omega Yeast; OYL 402) was added at 10 million cells/ml/° P (15 million cells/ml). The fermentation proceeded in 50 litre volumes (air lock/anaerobic) for 2 weeks. Fermentation volumes were 50 litre and were anaerobic with air locks at 70-72° F. After 14 days of fermentation, samples were transferred off of the yeast slurry to assess. Samples were decanted and stored at −80° C. in 50 ml conical tubes with the addition of sodium metabisulfite, to assist in preventing oxidation of thiols. In parallel, 50 ml was frozen with 15 ppm sodium metabisulfite. Where wort precursor levels were measured, samples were frozen pre-fermentation after the whirlpool step. Samples were sent for UPLC-MS/MS analysis, as above.

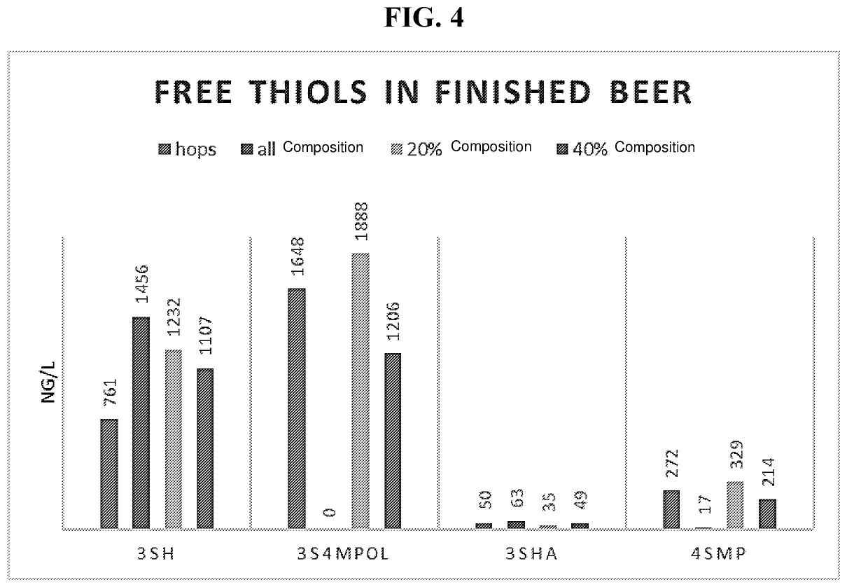

Results are shown in FIG. 4 and in Tables 9 and 10. For Tables 9 and 10, measurements are shown for the following: (1) not carb=finished beer that has been fully fermented, but not carbonated; (2) carb=finished beer that has been carbonated; (3) knockout=wort (unfermented beer) being transferred from the whirlpool into the fermenter; (4) pre-WP=wort in the kettle stage, before it is transferred to the whirlpool; (5) post-WP=after the whirlpool additions.

Notably, the use of grape skin composition without hops produced substantially higher 3MH levels in the beer, when compared to the 3MH levels produced by hops alone (FIG. 4). Moreover, the addition of only 20% w/w grape skin composition with 80% w/w hops significantly boosted 3MH levels in the beer compared to the 3MH levels observed when hops were used alone (FIG. 4). Without wishing to be bound by theory, it was proposed that there may be an entourage effect, whereby a small amount of grape skin composition introduced relative to the hops dose can dramatically increase the overall 3MH levels in the fermented beverage.

Surprisingly, the grape skin composition appeared to reduce 3M4MP and 4MMP levels in a dose dependent manner in the beer (FIG. 4, Table 9). This is highly advantageous given that high levels of 3M4MP and 4MMP can produce unpleasant sensory characteristics in fermented beverages such as beer, e.g., cat urine odour. It was noted also that CG3MH precursor levels were dramatically reduced in the beer by addition of the grape skin composition (Table 10). This was unexpected given the significant increases in 3MH levels observed in the beer.

TABLE 9
Volatile thiols in brewing samples

| | | | | | 3-sulfanyl-4- | | 4-methyl-4- |
|---|---|---|---|---|---|---|---|
| | | | | 3-sulfanylhexan- | methylpentan- | 3-sulfanylhexan- | sulfanylpentan- |
| | | | | 1-ol | 1-ol | 1-ol acetate | 2-one |
| | | | | 3SH | 3S4MPol | 3SHA | 4SMP |
| | | | Volume/ | (3MH) | (3M4MP) | (3MHA) | (4MMP) |
| Sample | Code | Material tested | Mass | (ng/L) | (ng/L) | (ng/L) | (ng/L) |
| 221316 | E1 | #22022 [0%] Knockout | 50 mL | 322 | 1359 | 53 | 384 |
| 221316 | E2 | #22022 [0%] Finished-carb | 50 mL | 716 | 1552 | 55 | 286 |
| 221316 | E3 | #22022 [0%] Finished - not carb | 50 mL | 805 | 1745 | 44 | 259 |
| 221316 | E4 | #22023 [100%] Knockout | 50 mL | 238 | nd | 48 | 2 |
| 221316 | E5 | #22023 [100%] Finished-carb | 50 mL | 1622 | nd | 71 | 17 |
| 221316 | E6 | #22023 [100%] Finished - not carb | 50 mL | 1290 | nd | 56 | 18 |
| 221316 | E7 | #22024 [20%] Knockout | 50 mL | 290 | 1061 | 29 | 282 |
| 221316 | E8 | #22024 [20%] Finished-carb | 50 mL | 1258 | 1957 | 34 | 323 |
| 221316 | E9 | #22024 [20%] Finished - not carb | 50 mL | 1205 | 1820 | 35 | 335 |
| 221316 | E10 | #22025 [40%] Knockout | 50 mL | 224 | 809 | 27 | 237 |
| 221316 | E11 | #22025 [40%] Finished-carb | 50 mL | 1100 | 1166 | 52 | 189 |
| 221316 | E12 | #22025 [40%] Finished - not carb | 50 mL | 1114 | 1247 | 46 | 239 |
| 221316 | E13 | #22022 [0%] 23 PRE-WP | 50 mL | 73 | nd | 36 | nd |
| 221316 | E14 | #22024 [20%] 25 PRE-WP | 50 mL | 81 | nd | 41 | 4 |

TABLE 10
Thiol precursors in brewing samples

| | | | | cysteine- | cysteinyl- | γ-glutamyl- | gluthation- | cysteine- | gluthation- |
|---|---|---|---|---|---|---|---|---|---|
| | | | | 3MH | glycine-3MH | cysteine-3MH | 3MH | 4MMP | 4MMP |
| | | | Volume/ | C3MH | CG3MH | GC3MH | G3MH | C4MMP | G4MMP |
| Sample | Code | Material tested | Mass | (μg/L) | (μg/L) | (μg/L) | (μg/L) | (μg/L) | (μg/L) |
| 221316 | E1 | #22022 [0%] Knockout | 50 mL | 9 | 102 | 22 | 136 | nd | 0.51 |
| 221316 | E2 | #22022 [0%] Finished-carb | 50 mL | 20 | 25 | 27 | 96 | nd | 0.49 |
| 221316 | E3 | #22022 [0%] Finished - not carb | 50 mL | 25 | 30 | 33 | 120 | 0.5 | 0.60 |
| 221316 | E4 | #22023 [100%] Knockout | 50 mL | 15 | 80 | 16 | 97 | nd | nd |
| 221316 | E5 | #22023 [100%] Finished-carb | 50 mL | 26 | 3 | 28 | 84 | nd | nd |
| 221316 | E6 | #22023 [100%] Finished - not carb | 50 mL | 26 | 3 | 28 | 87 | nd | nd |
| 221316 | E7 | #22024 [20%] Knockout | 50 mL | 10 | 101 | 23 | 151 | 0.3 | 0.5 |
| 221316 | E8 | #22024 [20%] Finished-carb | 50 mL | 33 | 3 | 56 | 91 | 0.3 | 0.6 |
| 221316 | E9 | #22024 [20%] Finished - not carb | 50 mL | 34 | 3 | 54 | 89 | 0.3 | 0.5 |
| 221316 | E10 | #22025 [40%] Knockout | 50 mL | 9 | 85 | 19 | 126 | 0.4 | 0.4 |
| 221316 | E11 | #22025 [40%] Finished-carb | 50 mL | 34 | 4 | 47 | 103 | 0.5 | 0.5 |
| 221316 | E12 | #22025 [40%] Finished - not carb | 50 mL | 34 | 4 | 47 | 102 | 0.5 | 0.5 |
| 221316 | E13 | #22022 [0%] 23 PRE-WP | 50 mL | 5 | 96 | 19 | 121 | nd | nd |
| 221316 | E14 | #22024 [20%] 25 PRE-WP | 50 mL | 4 | 87 | 16 | 109 | nd | nd |

TABLE 11
Sensory information for beer samples

| | | | Extract | |
|---|---|---|---|---|
| Flask sample | Yeast Strain | Treatment | (g/L) | Sensory |
| 1 - | OYL-402 | n/a | n/a | Moderate haze. Aroma: Moderate sulphur, grain, fruity esters. |
| Control | | | | Taste: Sulphur that adds a sharpness, slight tropical note. |
| Lot 3 | OYL-402 | Grape skin | 8 | Moderate haze. Aroma: Some sulfur, not as much as control. |
| 8 g/L | | powder | | Taste: Tropical notes, raisin, pleasant acidity. |
| Lot 3 | OYL-402 | Grape skin | 16 | Extremely hazy. Aroma: Strong aroma, somehow eggy without |
| 16 g/L | | powder | | a lot of sulfur? Raisin, smells oxidized (does not look |
| | | | | oxidized), maybe some apple. Taste: Intense. Acidic, drying |
| | | | | (tannic?), pronounced raisin, slight bitterness, melon. |
| 21-750-02 | OYL-402 | Grape skin | 8 | Moderate haze. Aroma: Moderate sulfur (eggy), fruity. |
| | | powder | | Taste: Slight raisin, slight bitterness, decent acidity, |
| | | | | grain, tropical notes. |
| 21-750-03 | OYL-402 | Grape skin | 8 | Moderate haze. Aroma: Tropical fruits, stale note. |
| | | powder | | Taste: Tastes oxidized (does not look oxidized), |
| | | | | red apple, some bitterness. |

TABLE 12
Thiol precursor and pH levels in wort samples

| Flask | Starting | Final | C3MH | CG3MH | GC3MH | G3MH | C4MMP | G4MMP |
|---|---|---|---|---|---|---|---|---|
| Sample | pH | pH | (μg/L) | (μg/L) | (μg/L) | (μg/L) | (μg/L) | (μg/L) |
| 1 - Control | 5.43 | 4.51 | 4 | 86.7 | 10.2 | 36.9 | nd | nd |
| Lot 3 8 g/L | 5 | 4.41 | 30.1 | 65.9 | 11.5 | 59.4 | nd | nd |
| Lot 3 16 g/L | 4.76 | 4.27 | 52.6 | 63.5 | 14.7 | 82.7 | nd | nd |
| 21-750-02 | 4.97 | 4.3 | 6.1 | 34.5 | 5.5 | 21.2 | nd | nd |
| 21-750-03 | 4.99 | 4.3 | 7.3 | 45.8 | 6.8 | 31.9 | nd | nd |

TABLE 13
Volatile thiol levels and specific gravity in beer samples

| | Starting | Final | 3SH | 3S4MPol | 3SHA | 4SMP |
|---|---|---|---|---|---|---|
| Flask | Gravity | Gravity | (3MH) | (3M4MP) | (3MHA) | (4MMP) |
| Sample | (°P) | (°P) | (ng/L) | (ng/L) | (ng/L) | (ng/L) |
| 1 - Control | 14.7 | 2.9 | 566 | nd | 42 | 3.3 |
| Lot 3 8 g/L | 15.1 | 2.9 | 1208 | nd | 24 | 4.7 |
| Lot 3 16 g/L | 15.4 | 2.7 | 2144 | nd | 27 | 15.9 |
| 21-750-02 | 15 | 2.9 | 1037 | nd | 20 | 4.3 |
| 21-750-03 | 15.2 | 2.7 | 1015 | nd | 21 | 5.9 |

Persons of ordinary skill can utilise the disclosures and teachings herein to produce other embodiments and variations without undue experimentation. All such embodiments and variations are considered to be part of this disclosure.

Accordingly, one of ordinary skill in the art will readily appreciate from the disclosure that later modifications, substitutions, and/or variations performing substantially the same function or achieving substantially the same result as embodiments described herein may be utilised according to such related embodiments. Thus, this disclosure is intended to encompass, within its scope, the modifications, substitutions, and variations to processes, manufactures, compositions of matter, compounds, means, methods, and/or steps set out herein.

In this specification, where reference has been made to external sources of information, including patent specifications and other documents, this is generally for the purpose of providing a context for discussing the features of this disclosure. Unless stated otherwise, reference to such sources of information is not to be construed, in any jurisdiction, as an admission that such sources of information are prior art or form part of the common general knowledge in the art.

The description herein may contain subject matter that falls outside of the scope of the claimed invention. This subject matter is included to aid understanding of the invention.

### REFERENCES

Anfang, N, Brajkovich, M, Goddard, M R. Co-fermentation with Pichia kluyveri increases varietal thiol concentrations in Sauvignon Blanc. Australian Journal of Grape Wine Research 15(1), 1-8, 2009.
Brajkovich, M, Peron, G, Lund, C M, Dykes, S I, Kilmartin, P A, Nicolau, L. Effect of screwcap and cork closures on SO₂ levels and aromas in a Sauvignon Blanc wine. Journal of Agricultural and Food Chemistry 53, 10006-10011, 2005.
Coetzee, C D T, du Toit, W J. A comprehensive review on Sauvignon blanc aroma with a focus on certain positive volatile thiols. Food Research International 45, 287-298, 2012.
Darriet, P, Tominaga, T, Lavigne, V, Boidron, J-N, Dubourdieu, D. Identification of a powerful aromatic component of Vitis vinifera L. var. Sauvignon wines: 4-mercapto-4-methylpentan-2-one. Flavour and Fragrance Journal 10(6), 385-392, 1995.
de Torres, C, Schumacher, R, Alañón, M E, Pérez-Coello, M S, Díaz-Maroto, M C. Freeze-dried grape skins by-products to enhance the quality of white wines from neutral grape varieties. Food Research International 69, 97-105, 2015.
Du Plessis, C S, Augustyn, O P H. Initial Study on the Guava Aroma of Chenin Blanc and Colombar Wines. South African Journal of Enology and Viticulture 2, 101, 1981.
Escalante, W D E. Perspectives and uses of non-saccharomyces yeasts in fermented beverages. Frontiers and New Trends in the Science of Fermented Food and Beverages, IntechOpen, DOI: http://dx.doi.org/10.5772/intechopen.81868
Holt, S, Miks, M H, de Carvalho, B T, Foulquie-Moreno, M R, Thevelein, J M. The molecular biology of fruity and floral aromas in beer and other alcoholic beverages. FEMS Microbiology Reviews 43, 193-222, 2019.
Jelley, R E, Herbst-Johnstone, M, Klaere, S, Pilkington, L I, Grose, C, Martin, D, Barker, D, Fedrizzi, B, Optimization of ecofriendly extraction of bioactive monomeric phenolics and useful flavor precursors from grape waste. ACS Sustainable Chemical Engineering 4, 5060-5067, 2016.
Jelley, R E, Deed, R C, Barker, D, Parish-Virtue, K, Fedrizzi, B. Fermentation of Sauvignon blanc grape marc extract yields important wine aroma 3-sulfanylhexan-1-ol (3SH). LWT—Food Science and Technology 131 109653, 2020.
Lund, C M T, Benkwitz, F, Wohler, M W, Triggs, C M, Gardner, R, Heymann, H, Nicolau, L. New Zealand Sauvignon Blanc Distinct Flavor Characteristics: Sensory, Chemical, and Consumer Aspects. American Journal of Enology and Viticulture 60, 1-12, 2009.
Maggu, M, Winz, R, Kilmartin, P A, Trought, M C T, Nicolau, L. effect of skin contact and pressure on the composition of sauvignon blanc must. Journal of Agricultural and Food Chemistry 55, 10281-10288, 2007.
Ribéreau-Gayon, P, Glories, Y, Maujean, A, Dubourdieu, D. Handbook of Enology: The chemistry of wine stabilization and treatments. John Wiley & Sons Ltd, Chicheste, 2006.
Robinson, J (ed) The Oxford Companion to Wine, Third Edition, Oxford University Press, 2006.
Roland, A, Schneider, R, Razungles, A, Cavelier, F. Varietal thiols in wine: discovery, analysis, and applications. Chemical Reviews 111, 7355-7376, 2011.
Spanos, G A and Wrolstad, R E. Influence of processing and storage on the phenolic composition of Thompson Seedless grape juice. Journal of Agricultural and Food Chemistry 38(7): 1565-1571, 1990.
Takoi, K, Degueil, M, Shinkaruk, S, Thibon, C, Maeda, K, Ito, K, Bennetau, B, Dubourdieu, D, Tominaga, T. Identification and characteristics of new volatile thiols derived from the Hop (Humulus lupulus L.) cultivar Nelson Sauvin. Journal of Agricultural and Food Chemistry 57, 2493-2502, 2009.
Tominaga, T, Dubourdieu, D. A New Type of Flavor Precursors in Vitis vinifera L cv. Sauvignon Blanc-S-Cysteine Conjugates. Journal of Agricultural and Food Chemistry 46, 5215-5219, 1998.
Tominaga, T, Furrer, A, Henry, R, Dubourdieu, D. Identification of new volatile thiols in the aroma of Vitis vinifera L. var. Sauvignon Blanc wines Flavour and Fragrance Journal 13, 159-162, 1998.
Tominaga, T, Darriet, P, Dubourdieu, D. Identification de l'acétate de 3-mercaptohexanol, composé à forte odeur de buis, intervenant dans l'arôme des vins de Sauvignon Vitis 35, 207-210, 1996.

What is claimed is:

1. A method for preparing a composition comprising a grape skin concentrate, the method comprising:
(i) obtaining skin of white wine grapes, such skin having flesh and juice components substantially removed,
(ii) freezing the skin of the white wine grapes,
(iii) thawing the skin of the white wine grapes,
(iv) drying the skin of the white wine grapes to produce a substantially dried product,
thereby obtaining a composition comprising a grape skin concentrate, and
combining the grape skin concentrate with one or more hops, one or more hops components, one or more yeast, one or more bacteria, and/or one or more enzymes.

2. The method of claim 1, wherein the white wine grapes are aromatic white wine grapes.

3. The method of claim 1 or claim 2, wherein the white wine grapes are selected from the group consisting of: white wine grapes from New Zealand, Australia, South Africa, South America, and any combination thereof.

4. The method of any one of claims 1 to 3, wherein the white wine grapes are selected from the group consisting of: Sauvignon Blanc, Albarino, Chenin Blanc, Colombard, Friulano, Tocai, Gruner Veltliner, Traminer, Verdicchio, Verdejo, Vermentino, Scheurebe, Maccabeo, Gewurztraminer, Riesling, Muscat, Petit Manseng, Pinot Gris, and Tokay grapes, and any combination thereof.

5. The method of any one of claims 1 to 3, wherein the white wine grapes comprise Sauvignon Blanc grapes.

6. The method of any one of claims 1 to 3, wherein (i) the white wine grapes consist essentially of Sauvignon Blanc grapes, or (ii) the white wine grapes are Sauvignon Blanc grapes.

7. The method of any one of claims 1 to 6, wherein the substantially removing of (i) comprises crushing or pressing.

8. The method of claim 7, wherein the pressing is performed by a pneumatic press or a basket press.

9. The method of claim 7, wherein the pressing is performed (i) with a press setting ranging from about 600 to about 800 litres per tonne, or (ii) with a press setting ranging from about 650 to about 720 litres per tonne.

10. The method of any one of claims 1 to 9, wherein the method includes addition of at least one antioxidant or preservative before or after (i).

11. The method of claim 10, wherein (i) the at least one antioxidant is a sulphite, or (ii) the at least one antioxidant is ascorbic acid.

12. The method of any one of claims 1 to 11, wherein the freezing of (ii) comprises blast freezing.

13. The method of any one of claims 1 to 11, wherein the freezing of (ii) comprises cryomaceration.

14. The method of any one of claims 1 to 13, wherein the drying of (iv) comprises freeze drying.

15. The method of any one of claims 1 to 14, wherein the method further comprises:
(a) milling the dried product of (iv) to obtain a milled product,
(b) formulating the dried product of (iv) to obtain a pellet product, or
(c) mixing the dried product of (iv) or the milled product of (a) with one or more liquids to obtain a liquid or semi-liquid product.

16. The method of claim 15, wherein the milled product comprises a flake or a powder.

17. The method of claim 15, wherein the composition is formulated to include one or more hops or hops components.

18. The method of claim 17, wherein:
(i) the one or more hops are selected from the group consisting of: American, Australian, English, German, New Zealand, and South African hops, and any combination thereof,
(ii) the one or more hops are selected from the group consisting of: Nelson Sauvin hops, Hallertau Blanc hops, Tomahawk hops, Simcoe hops, Summit hops, Cascade hops, and any combination thereof, and/or
(iii) the one or more hops are selected from the group consisting of: Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Nectaron, Saaz-CZ, Talus, Vista, Ella, Enigma, and Vic Secret hops, and any combination thereof.

19. The method of claim 17, wherein (i) the one or more hops components are selected from the group consisting of lupulin, hops terpenes, and hops esters, (ii) the one or more hops or hops components include cryogenically prepared hops, or (iii) the one or more hops or hops components include lupulin.

20. A composition comprising a grape skin concentrate, the composition being prepared according to any one of claims 1 to 19.

21. The composition of claim 20, wherein (i) the composition comprises a milled form, (ii) the composition comprises a flake form or a powder form, (iii) the composition comprises a pellet form, or (iv) the composition comprises a liquid form or a semi-liquid form.

22. The composition of claim 20 or 21, wherein (i) the composition further comprises one or more yeast, (ii) the composition further comprises one or more bacteria, (iii) the composition further comprises one or more enzymes, and/or (iv) the composition further comprises one or more hops or hops components.

23. The composition of claim 22, wherein:
(i) the one or more yeast are selected from the group consisting of: Saccharomyces, Brettanomyces, Kloeckera, Candida, Hanseniaspora, Pichia yeast, and any combination thereof,
(ii) the one or more yeast include a Pichia kluyveri yeast,
(iii) the one or more yeast include a Saccharomyces cerevisiae yeast,
(iv) the one or more yeast include a brewer's yeast, and/or
(v) the one or more yeast include yeast used for conversion of thiol precursors into free thiols.

24. The composition of claim 22 or claim 23, wherein:
(i) the one or more bacteria are selected from the group consisting of: Lactobacillus bacteria, Pediococcus bacteria, and any combination thereof,
(ii) the one or more bacteria are selected from the group consisting of: Lactobacillus delbrueckii, Lactobacillus plantarum, Pediococcus damnosus, and any combination thereof, and/or
(iii) the one or more bacteria include a bacteria used for lactic acid production.

25. The composition of any one of claims 22 to 24, wherein:
(i) the one or more hops include cryogenically prepared hops,
(ii) the one or more hops components include lupulin,
(iii) the one or more hops components include hops terpenes, and/or
(iv) the one or more hops components include hops esters,

26. The composition of any one of claims 22 to 25, wherein:
(i) the one or more enzymes include β-lyase, and/or
(ii) the one or more enzymes include β-glucosidase

27. The composition of any one of claims 22 to 26, wherein:
(i) the one or more hops are selected from the group consisting of: American, Australian, English, German, New Zealand, and South African hops, and any combination thereof,
(ii) wherein the one or more hops are selected from the group consisting of: Nelson Sauvin hops, Hallertau Blanc hops, Tomahawk hops, Simcoe hops, Summit hops, Cascade hops, and any combination thereof, and/or
(iii) wherein the one or more hops are selected from the group consisting of: Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Nectaron, Saaz-CZ, Talus, Vista, Ella, Enigma, and Vic Secret hops, and any combination thereof.

28. A composition comprising a grape skin concentrate and at least one additional ingredient, the composition being prepared according to any one of claims 1 to 19.

29. The composition of claim 28, wherein (i) the composition comprises a milled form, (ii) the composition comprises a flake form or a powder form, (iii) the composition comprises a pellet form, or (iv) the composition comprises a liquid form or a semi-liquid form.

30. The composition of claim 28 or claim 29, wherein (i) the at least one additional ingredient includes one or more yeast, (ii) the at least one additional ingredient includes one or more bacteria, (iii) the at least one additional ingredient includes one or more enzymes, and/or (iv) the at least one additional ingredient includes one or more hops or hops components.

31. The composition of claim 30, wherein:
(i) the one or more yeast are selected from the group consisting of: Saccharomyces, Brettanomyces, Kloeckera, Candida, Hanseniaspora, Pichia yeast, and any combination thereof,
(ii) the one or more yeast include a Pichia kluyveri yeast,
(iii) the one or more yeast include a Saccharomyces cerevisiae yeast,
(iv) the one or more yeast include a brewer's yeast, and/or
(v) the one or more yeast include yeast used for conversion of thiol precursors into free thiols.

32. The composition of claim 30 or claim 31, wherein:
(i) the one or more bacteria are selected from the group consisting of: Lactobacillus bacteria, Pediococcus bacteria, and any combination thereof,
(ii) the one or more bacteria are selected from the group consisting of: Lactobacillus delbrueckii, Lactobacillus plantarum, Pediococcus damnosus, and any combination thereof, and/or
(iii) the one or more bacteria include a bacteria used for lactic acid production.

33. The composition of any one of claims 30 to 32, wherein:
(i) the one or more hops include cryogenically prepared hops,
(ii) the one or more hops components include lupulin,
(iii) the one or more hops components include hops terpenes, and/or
(iv) the one or more hops components include hops esters.

34. The composition of any one of claims 30 to 33, wherein:
(i) the one or more enzymes include β-lyase, and/or
(ii) the one or more enzymes include β-glucosidase.

35. The composition of any one of claims 30 to 34, wherein:
(i) the one or more hops are selected from the group consisting of: American, Australian, English, German, New Zealand, and South African hops, and any combination thereof,
(ii) wherein the one or more hops are selected from the group consisting of: Nelson Sauvin hops, Hallertau Blanc hops, Tomahawk hops, Simcoe hops, Summit hops, Cascade hops, and any combination thereof, and/or
(iii) wherein the one or more hops are selected from the group consisting of: Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Nectaron, Saaz-CZ, Talus, Vista, Ella, Enigma, and Vic Secret hops, and any combination thereof.

36. A method of making a fermented beverage, comprising:
introducing a composition comprising a grape skin concentrate during a fermentation and/or prior to initiation of a fermentation,
wherein the composition comprising the grape skin concentrate is prepared according to any one of claims 1 to 19, and
carrying out the fermentation, thereby obtaining the fermented beverage.

37. The method of claim 36, further comprising introducing the composition post-fermentation.

38. The method of claim 36 or claim 37, wherein (i) the composition comprises a milled form, (ii) the composition comprises a flake form or a powder form, (iii) the composition comprises a pellet form, or (iv) the composition comprises a liquid form or a semi-liquid form.

39. The method of any one of claims 36 to 38, wherein:
(i) the method includes introducing one or more yeast during the fermentation and/or prior to initiation of the fermentation,
(ii) the method includes introducing one or more bacteria during the fermentation and/or prior to initiation of the fermentation,
(iii) the method includes introducing one or more enzymes during the fermentation and/or prior to initiation of the fermentation, and/or
(iv) the method includes introducing one or more hops or hops components during the fermentation, prior to initiation of the fermentation, and/or after the fermentation.

40. The method of any one of claims 36 to 39, wherein (i) the composition includes one or more yeast, (ii) the composition includes one or more bacteria, (iii) the composition includes one or more enzymes, and/or (iv) the composition includes one or more hops or hops components.

41. The method of claim 39 or claim 40, wherein:
(i) the one or more yeast are selected from the group consisting of: Saccharomyces, Brettanomyces, Kloeckera, Candida, Hanseniaspora, Pichia yeast, and any combination thereof,
(ii) the one or more yeast include a Pichia kluyveri yeast,
(iii) the one or more yeast include a Saccharomyces cerevisiae yeast,
(iv) the one or more yeast include a brewer's yeast, and/or
(v) the one or more yeast include yeast used for conversion of thiol precursors into free thiols.

42. The method of any one of claims 39 to 41, wherein:
(i) the one or more bacteria are selected from the group consisting of: Lactobacillus bacteria, Pediococcus bacteria, and any combination thereof,
(ii) the one or more bacteria are selected from the group consisting of: Lactobacillus delbrueckii, Lactobacillus plantarum, Pediococcus damnosus, and any combination thereof, and/or
(iii) the one or more bacteria include a bacteria used for lactic acid production.

43. The method of any one of claims 39 to 42, wherein:
(i) the one or more hops include cryogenically prepared hops,
(ii) the one or more hops components include lupulin,
(iii) the one or more hops components include hops terpenes, and/or
(iv) the one or more hops components include hops esters.

44. The method of any one of claims 39 to 43, wherein:
(i) the one or more enzymes include β-lyase, and/or
(ii) the one or more enzymes include β-glucosidase.

45. The method of any one of claims 39 to 44, wherein:
(i) the one or more hops are selected from the group consisting of: American, Australian, English, German, New Zealand, and South African hops, and any combination thereof,
(ii) wherein the one or more hops are selected from the group consisting of: Nelson Sauvin hops, Hallertau Blanc hops, Tomahawk hops, Simcoe hops, Summit hops, Cascade hops, and any combination thereof, and/or
(iii) wherein the one or more hops are selected from the group consisting of: Citra, Chinook, Loral, Mosaic, Amarillo, Centennial, Ekuanot, Sabro, Southern Cross, Pacific Gem, Pacific Jade, Riwaka, Dr Rudi, Wakatu, Wai iti, Waimea, Kohatu, Motueka, Green Bullet, Hort 4337, Hort 9909, Orbit, Rakau, Pacifica, Styrian Golding, Strata, Galaxy, Calypso, El Dorado, Hallertauer Mittelfrüher, Idaho 7, Nectaron, Saaz-CZ, Talus, Vista, Ella, Enigma, and Vic Secret hops, and any combination thereof.

46. The method of any one of claims 36 to 45, wherein the fermented beverage is an alcoholic, non-alcoholic, or reduced-alcohol beverage.

47. The method of any one of claims 36 to 46, wherein:
(i) the fermented beverage is a beer,
(ii) the fermented beverage is selected from the group consisting of: barley or other cereal beers, maize beers, millet beers, oat beers, rice beers, rye beers, sorghum beers, and wheat beers, and/or
(iii) the fermented beverage is selected from the group consisting of: ales, lagers, pilsners, porters, saisons, and stouts.

48. The method of claim 47, wherein:
(i) the beer is an India Pale Ale (IPA) beer,
(ii) the beer is a New England IPA, and/or
(iii) the beer is a hazy IPA.

49. The method of any one of claims 36 to 46, wherein:
(i) the fermented beverage is a beer-wine hybrid,
(ii) the fermented beverage is a fruit beer,
(iii) the fermented beverage is a cider, or
(iv) the fermented beverage is a kombucha.

50. A fermented beverage prepared using the method according to any one of claims 36 to 49.

51. The fermented beverage of claim 50, wherein the fermented beverage is an alcoholic, non-alcoholic, or reduced-alcohol beverage.

52. The fermented beverage of claim 50 or claim 51, wherein:
(i) the fermented beverage is a beer,
(ii) the fermented beverage is selected from the group consisting of: barley or other cereal beers, maize beers, millet beers, oat beers, rice beers, rye beers, sorghum beers, and wheat beers, and/or
(iii) the fermented beverage is selected from the group consisting of: ales, lagers, pilsners, porters, saisons, and stouts.

53. The fermented beverage of claim 52, wherein:
(i) the beer is an India Pale Ale (IPA) beer,
(ii) the beer is a New England IPA, and/or
(iii) the beer is a hazy IPA.

54. The fermented beverage of claim 50 or claim 51, wherein:
(i) the fermented beverage is a beer-wine hybrid,
(ii) the fermented beverage is a fruit beer,
(iii) the fermented beverage is a cider, or
(iv) the fermented beverage is a kombucha.
